(19) United States
(12) Patent Application Publication
(10) Pub. No.: US 2022/0389409 A1
(43) Pub. Date: Dec. 8, 2022

(54) HIGH-RESOLUTION SPATIAL AND QUANTITATIVE DNA ASSESSMENT

(71) Applicants: THE BROAD INSTITUTE, INC., Cambridge, MA (US); PRESIDENT AND FELLOWS OF HARVARD COLLEGE, Cambridge, MA (US); THE GENERAL HOSPITAL CORPORATION, Boston, MA (US)

(72) Inventors: Tongtong Zhao, Cambridge, MA (US); Fei Chen, Cambridge, MA (US); Jason Buenrostro, Cambridge, MA (US); Evan Macosko, Boston, MA (US)

(73) Assignees: THE BROAD INSTITUTE, INC., Cambridge, MA (US); PRESIDENT AND FELLOWS OF HARVARD COLLEGE, Cambridge, MA (US); THE GENERAL HOSPITAL CORPORATION, Boston, MA (US)

(21) Appl. No.: 17/775,471

(22) PCT Filed: Nov. 9, 2020
(86) PCT No.: PCT/US2020/059728

§ 371 Date: May 9, 2022

Related U.S. Application Data
(60) Provisional application No. 62/933,794, filed on Nov. 11, 2019.

(51) Int. Cl. C12N 15/10; C12Q 1/6806

(52) U.S. Cl. CPC C12N 15/1065; C12N 15/1017; C12Q 1/6806

(57) ABSTRACT

The present disclosure relates to compositions and methods for assessing relative DNA levels (e.g., levels of genomic DNA, mtDNA, viral DNA, bacterial DNA, etc.) in a spatially-defined manner across a tissue sample, specifically providing DNA sequence identity and relative abundance information at high-resolution across multiple locations assessed across the tissue sample.

### CROSS REFERENCE TO RELATED APPLICATION

This application claims the benefit of U.S. Provisional Application No. 62/933,794, filed Nov. 11, 2019, entitled “High-Resolution Spatial and Quantitative DNA Assessment.” The entire contents of the aforementioned application are incorporated herein by reference.

### STATEMENT REGARDING FEDERALLY SPONSORED RESEARCH

This invention was made with government support under Grant No. 1DP5OD024583, awarded by the National Institutes of Health. The government has certain rights in the invention.

### FIELD OF THE INVENTION

The invention relates generally to methods and compositions for spatial assessment of DNA abundance in a tissue sample.

### SEQUENCE LISTING

The instant application contains a Sequence Listing which has been filed electronically in ASCII format and is hereby incorporated by reference in its entirety. Said ASCII copy, created on Nov. 9, 2020, is named 52199_551001WO_BI10588_SL.txt and is 4 kB in size.

### BACKGROUND OF THE INVENTION

Approaches for spatial monitoring of DNA in a tissue sample include traditional histological approaches, in which sections of tissue are fixed, stained, and assessed, e.g., for the presence of probe(s) and/or label across the viewable region of the fixed tissue section on a microscope slide, as well as certain more recent in situ techniques for macromolecule monitoring that can be applied to detection of DNA, which in many cases have been afflicted by being laborious in application, offering a low degree of multiplexing with a high degree of technical difficulty and/or providing only low resolution of spatial capture across an array (i.e., providing only approximately 100−200 μm resolution). An approach for spatial monitoring of macromolecule abundance at near-single cell levels of resolution has recently been described in PCT/US19/30194. A need exists for improved approaches for providing spatial DNA abundance data (e.g., to allow for detection of copy number variation (CNV), enhance cell lineage identification, etc.) at resolutions approaching single cell resolution.

BRIEF SUMMARY OF THE INVENTION

The current disclosure relates, at least in part, to compositions and methods for assessing DNA abundance (e.g., nuclear/genomic DNA, e.g., to allow detection of copy number variation (CNV) and/or cell lineage; mtDNA; viral/retroviral DNA; bacterial DNA, etc.) in a tissue sample, which provide deep sequence coverage at high-resolution across multiple locations assessed within a tissue sample. Specifically contemplated applications for such improved, high-resolution spatial assessment of DNA abundance include, but are not limited to, assessment of copy number variation (CNV), mitochondrial lineage tracing, assessment of epigenetic regulation, identification of regions of monoallelic gene expression and gene dosage in an assayed tissue, evaluation of gene therapy deliverables to tissue, including, e.g., identification of cellular delivery of CRISPR/Cas9 plasmid(s) and/or gels, TALEN plasmid(s) and/or gels, viral vectors (e.g., AAV), expression vectors/plasmids in general, etc., and assessment of synthetic DNA arrays for sequence-specific quantities and distributions of DNA upon the synthetic DNA array. A wide range of diagnostic, therapeutic and research applications are therefore contemplated.

In one aspect, the instant disclosure provides a method for obtaining spatially-resolvable DNA abundance data from a tissue sample, the method involving (i) obtaining a tissue sample from a subject; (ii) preparing a section of the tissue sample; (iii) contacting the section of the tissue sample with a permeabilizing agent and a DNA fragmenting agent, thereby producing a treated sectioned tissue sample; (iv) obtaining a solid support; (v) contacting the solid support with a capture material, thereby forming a capture material-coated solid support; (vi) contacting the capture material—coated solid support with a population of 1-100 μm diameter beads, where each bead has at least 1000 attached oligonucleotides and where at least 1000 attached oligonucleotides of each bead each includes: (a) a bead identification sequence that is common to all at least 1000 oligonucleotides on each bead and (b) a target DNA-specific capture sequence, thereby forming a subpopulation of beads attached to the solid support; (vii) identifying the bead identification sequence and associated two-dimensional position on the solid support of individual beads of the subpopulation of beads attached to the solid support; (viii) contacting the subpopulation of 1-100 μm diameter beads captured upon the solid support with the treated sectioned tissue sample; and (ix) obtaining the sequences of a population of target DNA molecules bound to the bead oligonucleotides and an associated bead identification sequence for each target DNA molecule sequenced, thereby obtaining spatially-resolvable DNA abundance data from the tissue sample.

In embodiments, the permeabilizing agent is Triton X-100, NP-40, methanol, acetone, Tween 20, saponin, Leucoperm™, and/or digitonin. In a related embodiment, the permeabilizing agent is about 0.01%-5%.Triton X-100.

In certain embodiments, contacting of the section of the tissue sample with the permeabilizing agent is performed for a duration of time between about 0.1 minute and about 30 minutes.

In some embodiments, the DNA fragmenting agent is a transposase; H₂O₂; a DNase; and/or sonication. Optionally, the DNase is a restriction endonuclease. In a related embodiment, the DNA fragmenting agent includes a Tn5 transposase enzyme. Optionally, the Tn5 transposase enzyme is present in a tagmentation buffer that includes adapater and mosaic oligonucleotides. In further related embodiments, the Tn5 enzyme in tagmentation buffer including adapater and mosaic oligonucleotides contacts the sectioned tissue sample for about 20 minutes to about 16 hours, optionally about 30 minutes to about two hours. Optionally, the Tn5 enzyme in tagmentation buffer including adapater and mosaic oligonucleotides contacts the sectioned tissue sample at between about 25° C. and about 55° C.

In certain embodiments, the section of the tissue sample is a cryosection.

In some embodiments, the target DNA molecules are genomic DNA molecules, mitochondrial DNA (mtDNA) molecules, viral DNA molecules (optionally retroviral DNA molecules or AAV DNA molecules) and/or bacterial DNA molecules.

In one embodiment, the target DNA molecules are genomic DNA molecules. Optionally, the genomic DNA molecules are enriched for accessible chromatin sequences, as compared to inaccessible chromatin sequences (e.g., genomic DNA sequences associated with nucleosomes and/or other forms of condensed chromatin).

In certain embodiments, the permeabilizing agent is about 0.1% to about 0.5% Triton X-100. Optionally, the contacting of the section of the tissue sample with the Triton X-100 is performed for a duration of time between about 10 minutes and about 60 minutes.

In embodiments, step (iii) further includes contacting the section of the tissue sample with a nucleosome disrupting agent. Optionally, the nucleosome disrupting agent is HCl, SDS and/or a protease/proteinase. In a related embodiment, the nucleosome disrupting agent is about 0.01N to about 0.5HCl. Optionally, contacting the section of the tissue sample with HCl is performed for about one to about 10 minutes.

In one embodiment, the nucleosome disrupting agent is about 0.1% to about 10% SDS. Optionally, contacting the section of the tissue sample with SDS is performed for about 5 to about 15 minutes. Optionally, at about 30° C. to about 70° C.

In certain embodiments, step (iii) further involves contacting the section of the tissue sample with proteinase K. Optionally, about one to about 20 μg/ml proteinase K. Optionally, for a duration of time of about 5 to about 15 minutes. Optionally, at about 25° C. to about 50° C.

In embodiments, the tissue sample is obtained from brain, lung, liver, kidney, pancreas, heart and/or gastrointestinal (GI) tract.

In some embodiments, the tissue sample is obtained from a tumor.

In embodiments, the subject is a mammal. Optionally, the subject is a human.

In certain embodiments, the spatially-resolvable DNA abundance data identifies regions of copy number variation (CNV) in the section of the tissue sample. Optionally, regions of genetic amplification are identified in the tissue sample. Optionally, regions of trisomy are identified.

In another embodiment, the spatially-resolvable DNA abundance data identifies regions of aneuploidy in the section of the tissue sample.

In some embodiments, the spatially-resolvable DNA abundance data identifies regions of related cellular lineage in the section of the tissue sample.

In one embodiment, the tissue sample is fixed. Optionally, the tissue sample is fixed with paraformaldehyde. Optionally, fixation is performed for 5-25 minutes. In a related embodiment, fixation is quenched with 100-500mM Tris-HCl.

In another embodiment, the solid support is a slide. Optionally, the solid support is a glass slide.

In certain embodiments, the capture material is applied as a liquid. Optionally, the capture material is applied using a brush or aerosol spray. In a related embodiment, the capture material is a liquid electrical tape. Optionally, the capture material dries to form a vinyl polymer. In related embodiments, the vinyl polymer is polyvinyl hexane.

In embodiments, the 1-100 μm diameter beads include porous polystyrene, porous polymethacrylate and/or polyacrylamide.

In some embodiments, the beads are 1-40 μm diameter beads. Optionally, the beads are 10 μm beads.

In one embodiment, the step of (vii) identifying the bead identification sequence and associated two-dimensional position on the solid support of individual beads of the subpopulation of beads attached to the solid support involves performance of a sequencing-by-ligation technique.

In some embodiments, the subpopulation of 1-100 μm diameter beads captured upon the solid support in step (viii) is maintained at a temperature of between about 4° C. and about 30° C. Optionally, a temperature of about 25° C. is maintained.

In another embodiment, step (viii) further involves contacting the subpopulation of 1-100 μm diameter beads captured upon the solid support with a wash solution. Optionally, the wash solution is a saline solution. Optionally, the solution includes between about 1M and about 3M NaCl. In a related embodiment, the wash solution is a saline-sodium citrate buffer that includes between about 1M and about 3M NaCl.

In embodiments, step (ix) obtaining the sequences of a population of DNA molecules bound to the bead oligonucleotides and an associated bead identification sequence for each target DNA molecule sequenced involves a next-generation sequencing approach. Optionally, the next-generation sequencing approach is solid-phase, reversible dye-terminator sequencing; massively parallel signature sequencing; pyro-sequencing; sequencing-by-ligation; ion semiconductor sequencing; Nanopore sequencing; and/or DNA nanoball sequencing. In related embodiments, the next-generation sequencing approach is solid-phase, reversible dye-terminator sequencing.

In some embodiments, the bead identification sequence and associated two-dimensional position on the solid support of individual beads of the subpopulation of beads attached to the solid support is registered in a computer.

In another embodiment, a method of the instant disclosure further comprising step (x) generating an image of the tissue sample that depicts the location(s) and relative abundance of one or more captured target DNAs within the sample. Optionally, the image is a two-dimensional image.

In certain embodiments, the spatially-resolvable DNA abundance data identifies one or more of the following features in the section of the tissue sample, including: a) mitochondrial lineage; b) epigenetic modification(s) and/or difference(s) in regions of chromatin accessibility across the section of the tissue sample; c) regions of monoallelic gene expression and/or gene dosage; d) cellular delivery of CRISPR/Cas9 plasmid(s) and/or gels, TALEN plasmid(s) and/or gels, viral vectors (e.g., AAV), expression vectors/plasmids and/or other gene therapy agents/payloads; e) histology; and/or 0 response in the tissue to a pre-administered drug or other agent, as compared to an appropriate control.

Another aspect of the instant disclosure provides a method for providing access to cellular DNA of a tissue sample in situ, the method involving: (i) obtaining a tissue sample from a subject; (ii) preparing a section of the tissue sample; and (iii) contacting the section of the tissue sample with a permeabilizing agent and a DNA fragmenting agent, thereby providing access to cellular DNA of the tissue sample in situ.

In embodiments, the disclosure further involves (iv) contacting the section of the tissue sample with an array of DNA capture probes. Optionally the section is contacted with a bead array possessing DNA capture probes. Optionally, the bead array possessing DNA capture probes is attached to a solid support. In some embodiments, the spatial locations upon the solid support of a selection of beads of the bead array are known upon contact of the bead array with the section of the tissue sample.

An additional aspect of the instant disclosure provides an improved method for obtaining spatial DNA abundance data from a tissue sample, where the improvement involves: i) obtaining a section of a tissue sample; ii) contacting the section of the tissue sample with a permeabilizing agent and a DNA fragmenting agent, thereby producing a treated section of the tissue sample; and iii) contacting the treated section of the tissue sample with a capture material—coated solid support that includes a population of beads where each bead has at least 1000 attached oligonucleotides and where each of the at least 1000 attached oligonucleotides of each bead includes: (a) a bead identification sequence that is common to all at least 1000 oligonucleotides on each bead and (b) a target DNA-specific capture sequence, thereby obtaining spatial DNA abundance data from the tissue sample.

In one embodiment, the target DNA-specific capture sequence anneals to an exogenously introduced adapter sequence in the target DNA of the section of the tissue sample.

In another embodiment, the target DNA-specific capture sequence anneals to one or more pre-selected target DNA sequences within the section of the tissue sample. Optionally, the target DNA-specific capture sequence anneals to one or more pre-selected endogenous target DNA sequences within the section of the tissue sample.

In certain embodiments, the target DNA-specific capture sequence anneals to one or more viral or bacterial target DNA sequences within the section of the tissue sample. Optionally, the viral target DNA sequences are adeno-associated virus (AAV) target DNA sequences.

In embodiments, one or more non-DNA macromolecules or small molecules are extracted and/or identified in the same section of the tissue sample or in one or more adjacent section(s) of the tissue sample.

Another aspect of the instant disclosure provides a kit that includes: (i) a permeabilizing agent; (ii) a DNA fragmenting agent; (iii) a capture material-coated solid support with a population of 1-100 μm diameter beads, where each bead has at least 1000 attached oligonucleotides and where at least 1000 attached oligonucleotides of each bead each includes: (a) a bead identification sequence that is common to all at least 1000 oligonucleotides on each bead and (b) a target DNA-specific capture sequence, thereby forming a subpopulation of beads attached to the solid support; and instructions for its use.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “array” refers to a population of features or sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells, beads arranged upon a flat surface (e.g., a slide), optionally beads captured upon a flat surface (e.g., a layer of beads adhered to or otherwise stably associated with a slide (e.g., a layer of beads adsorbed to a slide-attached elastomeric surface)), etc.

As used herein, the term “attached” refers to the state of two things being joined, fastened, adhered, connected or bound to each other. For example, an analyte, such as a nucleic acid, can be attached to a material, such as a gel or solid support, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “barcode sequence” is intended to mean a series of nucleotides in a nucleic acid that can be used to identify the nucleic acid, a characteristic of the nucleic acid (e.g., the identity and optionally the location of a bead to which the nucleic acid is attached), or a manipulation that has been carried out on the nucleic acid. The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population (e.g., all nucleic acid species attached to a single bead might possess the same barcode sequence, while different beads present a different shared barcode sequence that serves to identify each such different bead). By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

As used herein, “beads”, “microbeads”, “microspheres” or “particles” or grammatical equivalents can include small discrete particles. The composition of the beads can vary, depending upon the class of capture probe, the method of synthesis, and other factors. In certain embodiments of the instant disclosure, the sizes of the beads of the instant disclosure tend to range from 1 μm to 100 μm in diameter (with all subranges within this range expressly contemplated), e.g., depending upon the extent of image resolution desired, nature of the solid support to be used for spatial bead array construction, sequencing processes (e.g., flow cell sequencing) to be employed, as well as other factors.

As used herein, the term “biological specimen” is intended to mean one or more cell, tissue, organism or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Target nucleic acids can also be derived from a prokaryote such as a bacterium, Escherichia coli, Staphylococci or Mycoplasma pneumoniae; an archae; a virus such as Hepatitis C virus or human immunodeficiency virus; or a viroid. Specimens can be derived from a homogeneous culture or population of the above organisms or alternatively from a collection of several different organisms, for example, in a community or ecosystem.

As used herein, the term “cleavage site” is intended to mean a location in a nucleic acid molecule that is susceptible to bond breakage. The location can be specific to a particular chemical, enzymatic or physical process that results in bond breakage. For example, the location can be a nucleotide that is abasic or a nucleotide that has a base that is susceptible to being removed to create an abasic site. Examples of nucleotides that are susceptible to being removed include uracil and 8-oxo-guanine as set forth in further detail herein below. The location can also be at or near a recognition sequence for a restriction endonuclease such as a nicking enzyme.

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “cryosection” refers to a piece of tissue, e.g. a biopsy, that has been obtained from a subject, snap frozen, embedded in optimal cutting temperature embedding material, frozen, and cut into thin sections. In certain embodiments, the thin sections can be treated with a permeabilizing agent and/or a DNA fragmenting agent, and then directly applied to an array of beads captured upon a solid support (e.g., a slide), or the thin sections can be fixed (e.g. in methanol or paraformaldehyde), treated with a permeabilizing agent and/or a DNA fragmenting agent, and then applied to a bead-presenting planar surface, e.g., a slide upon which a layer of microbeads has been attached/arrayed.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. Two beads can be different from each other by virtue of being attached to different nucleic acids.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “extend,” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended.

As used herein, the term “feature” means a location in an array for a particular species of molecule. A feature can contain only a single molecule or it can contain a population of several molecules of the same species. Features of an array are typically discrete. The discrete features can be contiguous or they can have spaces between each other. The size of the features and/or spacing between the features can vary such that arrays can be high density, medium density or lower density. High density arrays are characterized as having sites separated by less than about 15 μm. Medium density arrays have sites separated by about 15 to 30 μm, while low density arrays have sites separated by greater than 30 μm. An array useful herein can have, for example, sites that are separated by less than 100 μm, 50 μm, 10 μm, 5 μm, 1 μm, or 0.5 μm. An apparatus or method of the present disclosure can be used to detect an array at a resolution sufficient to distinguish sites at the above densities or density ranges.

The terms “isolated,” “purified, ” or “biologically pure” refer to material that is free to varying degrees from components which normally accompany it as found in its native state. “Isolate” denotes a degree of separation from original source or surroundings. “Purify” denotes a degree of separation that is higher than isolation.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid- phase, reversible dye-terminator sequencing (Solexa/Illumina™); SOLiD™ technology (Applied Biosystems); Ion semiconductor sequencing (Ion Torrent™); and DNA nanoball sequencing (Complete Genomics). Descriptions of certain NGS platforms can be found in the following: Shendure, er al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141 ; Su, et al., “Next-generation sequencing and its applications in molecular diagnostics” Expert Rev Mol Diagn, 2011 , 11 (3):333-43; and Zhang et al., “The impact of next-generation sequencing on genomics”, J Genet Genomics, 201, 38(3): 95-109.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid or sequence of a nucleic acid, are intended as semantic identifiers for the nucleic acid or sequence in the context of a method or composition set forth herein and does not necessarily limit the structure or function of the nucleic acid or sequence beyond what is otherwise explicitly indicated. The terms “probe” and “target” can be similarly applied to other analytes such as proteins, small molecules, cells or the like.

As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the species of analyte (e.g., nucleic acid of a particular sequence) that occupies each feature independent of whether spacing produces a random pattern or ordered pattern. An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Particularly useful solid supports for some embodiments are slides and beads capable of assorting/packing upon the surface of a slide (e.g., beads to which a large number of oligonucleotides are attached).

As used herein, the term “spatial tag” is intended to mean a nucleic acid having a sequence that is indicative of a location. Typically, the nucleic acid is a synthetic molecule having a sequence that is not found in one or more biological specimen that will be used with the nucleic acid. However, in some embodiments the nucleic acid molecule can be naturally derived or the sequence of the nucleic acid can be naturally occurring, for example, in a biological specimen that is used with the nucleic acid. The location indicated by a spatial tag can be a location in or on a biological specimen, in or on a solid support or a combination thereof. A barcode sequence can function as a spatial tag. In certain embodiments, the identification of the tag that serves as a spatial tag is only determined after a population of beads (each possessing a distinct barcode sequence) has been arrayed upon a solid support (optionally randomly arrayed upon a solid support) and sequencing of such a bead-associated barcode sequence has been determined in situ upon the solid support.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal and connective tissues.

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 depicts a schematic showing exemplary synthesis of a barcoded capture array, where individual bead location within an array affixed to a glass slide is identified via in situ sequencing and registered.

FIGS. 2A and 2B show a process of the instant disclosure for obtaining spatially resolvable genomic DNA sequencing in situ. FIG. 2A depicts an embodiment of the instant disclosure in which tissue is exposed to in situ tagmentation, to capture accessible genomic DNA for high throughput sequencing. Shown to the right of the tissue is a diagram depicting that nucleosomes are present and that accessible genomic DNA is assayed. Tn5 tagmentation is then applied, in which the Tn5 transposome randomly cleaves accessible double stranded DNA into fragments and tags the DNA, by end-joining to the 5-ends, synthetic “mosaic ends.” The mosaic ends, or Tn5 adapter sequences, complement bead adapter and Illumina® Handle sequences. On the far right is a schematic of the Slide-seq array: the tissue containing in situ tagged DNA is then pressed against a puck containing the Slide-seq array. In the array, beads 10 μm in diameter are bound to DNA sequences containing a “P7” primer binding sequence, a “BC” bar code sequence (for identifying/tracking the position of the bead), and a “Read 2” sequence which binds to the Tn5 bead adapter. The bound DNA is then released via photocleavage from the beads, and is subsequently amplified. The final genome library, ready to be sequenced, is composed of fragments as shown at the bottom of FIG. 2A. FIG. 2B shows, at left, the fragment counts as a function of distance from transcription start site (TSS). As expected for capturing open chromatin, fragments are enriched around TSS. At right, FIG. 2B shows counts of the insert sizes of assayed fragments that were generated. As expected for capturing open chromatin, the distribution displays nucleosome-associated periodicity.

FIGS. 3A to 3E show the results of tagmentation-directed DNA sequencing performed in situ. FIG. 3A shows a histogram of edit distance of mapped barcodes to Illumina™ sequencing of the same beads (blue (most highly represented in edit distance bins “1” and “2”) is the set of sequenced barcodes, vs orange (most highly represented in edit distance bins “3”, “4” and “5”) for scrambled barcodes). FIG. 3B, at left, shows the number of unique nuclear sequence reads per bead. FIG. 3B, at right, shows the number of unique mitochondrial sequence reads per bead. FIG. 3C shows the normalized genome coverage of a lung tumor section in terms of gene copy numbers represented per chromosome. The displayed Chromosome 6 data indicates trisomy, based on the copy numbers present. FIG. 3D, at left, shows a DAPI stained tissue slice extracted from the cerebellum. FIG. 3D, at right, shows the same tissue slice reconstructed based on the ratio of mitochondrial to nuclear sequence reads, which demonstrated the accuracy and resolution of the approach disclosed herein (reconstructed bead position vs actual cell position is shown). FIG. 3E shows a spatial depiction of observed coverage for individual chromosomes for z-scores, aggregated over 100 μm.

### DETAILED DESCRIPTION OF THE INVENTION

The present disclosure is directed, at least in part, to the discovery that high-resolution spatial DNA abundance data (e.g., nuclear/genomic DNA abundance; mtDNA; viral/retroviral DNA; bacterial DNA, etc.) can be robustly obtained from a tissue sample that has been treated with a permeabilizing agent and a DNA fragmenting agent. The disclosure therefore allows for deep sequence coverage to be obtained at high-resolution across multiple locations assessed within a tissue sample. Examples of contemplated applications for such improved, high-resolution spatial assessment of DNA abundance include, but are not limited to, assessment of copy number variation (CNV), mitochondrial lineage tracing, assessment of epigenetic regulation, identification of regions of monoallelic gene expression and gene dosage in an assayed tissue, evaluation of gene therapy deliverables to tissue, including, e.g., identification of cellular delivery of CRISPR/Cas9 plasmid(s) and/or gels, TALEN plasmid(s) and/or gels, viral vectors (e.g., AAV), expression vectors/plasmids in general, etc., as well as assessment of synthetic DNA arrays for sequence-specific quantities and distributions of DNA upon the synthetic DNA array.

The instant disclosure expands upon the content of PCT/US19/30194, and certain aspects of the instant disclosure specifically employ a tightly packed spatially barcoded microbead array (e.g., an array of 10 μm diameter beads packed at an inter-bead spacing of 20 μm or less, where each bead possesses a bead-specific barcode within bead-attached capture oligonucleotides) created via application of a capture material to a solid support (e.g., application of a liquid electrical tape to a glass slide, followed by application of a layer of microbeads), which can be used to capture cellular target DNA molecules of cryosectioned tissue, in a manner that is both spatially resolvable at high resolution (e.g., at resolutions of 20 μm between image features) and with deep coverage (i.e., high-resolution images of relative levels for individual target DNA sequences can be generated using the methods and compositions of the instant disclosure, for a large number (i.e., tens, hundreds or even thousands) of DNA sequences, across an individual cryosectioned tissue sample).

The instant disclosure enables spatially resolved capture of DNA for sequencing from cells and tissues with approximate 10 μm (single cell) resolution. Art-recognized spatial profiling technologies have primarily been directed to either targeted in situ techniques, which have been laborious and have offered only a low degree of multiplexing with a high degree of technical difficulty, or have offered only very low resolution on spatial capture arrays (resolutions of approximately 100−200 μm). The instant disclosure provides a level of image resolution that is a full order of magnitude superior in lateral resolution to such prior approaches, and two orders of magnitude superior in capture area. By using DNA capture and subsequent high-throughput sequencing (Illumina™ bead-based sequencing as exemplified herein), the instant disclosure provides methods and compositions that are easily adoptable and allows for whole and partial genomic profiling of complex tissues.

One key concept of the compositions and methods described herein is use of a spatially barcoded array of oligonucleotide-laden beads to capture DNA from tissue sections. Exemplified beads are synthesized with a unique or sufficiently unique bead barcode as previously described, e.g., in WO 2016/040476 (PCT/US2015/049178), where an exemplary sufficiently unique bead barcode is one that is a member of a population of barcode sequences that is sufficiently degenerate to a population (e.g., of beads) that a majority of individual components (e.g. beads) of the barcoded population each possesses a unique barcode sequence, where the remainder (minority) of the population may possess barcodes that are redundant with those of other members within the remainder population, yet such redundancy can either be eliminated or otherwise adjusted for (e.g., normalized, averaged across/between redundant members, etc.) with only minor impact upon, e.g., the image resolution obtained when employing such a barcoded population. Certain aspects of the instant disclosure employ the following: 1) tiling of beads into a monolayer surface; 2) interrogation of the sequence of each bead barcode of the surface via sequencing by ligation on an standard microscope; 3) capture of DNA from permeabilized and DNA-fragmented cells and tissues onto the bead array, noting the instant use of cryosectioned tissue samples; 4) generating barcoded sequencing libraries as previously described in WO 2016/040476; and 5) next-generation sequencing of the barcoded libraries (exemplified herein using an Illumina™ platform) followed by bead barcode matching to the spatial location of the read. Generation of high-resolution barcoded arrays via on-surface sequencing of capture probe beads (noting that exemplified beads have been prepared as previously described in WO 2016/040476) is a distinguishing feature of the instant disclsoure, as well as techniques to capture DNA to the barcoded bead array.

Various expressly contemplated components of certain compositions and methods of the instant disclosure are considered in additional detail below.

Understanding tissue function is facilitated not only by knowledge of cell types and states but also their spatial organization within the tissue. Current technologies to investigate tissue biology fall typically into one of two categories: 1) high throughput transcriptomics and/or (epi)genomics, which captures a comprehensive snapshot of cell states within a tissue but loses the spatial context of those cells, or 2) imaging-based methods that provide spatial information but are typically laborious and require a priori knowledge of which genes/transcripts/proteins to target. There is a need for improved technologies/platforms that allow unbiased, high-throughput capture of particularly genomic and/or mitochondrial DNA fragments from intact tissue while preserving their spatial context.

Examples of previous attempts to integrate genomics with spatial information have relied on physically separating regions of the tissue with laser capture microdissection (LCM), followed by next-generation sequencing. While this approach has provided many useful insights, its power is limited by low throughput (dissection takes time) and the inevitable destruction of parts of the tissue by the laser.

Two separate recent approaches, named Spatial Transcriptomics and “Slide-seq” (see, e.g., PCT/US19/30194), have provided for unbiased high-throughput capture of macromolecules, particularly transcriptome, with high spatial resolution. However, even with such approaches available, development of an improved means for capture of genomic and/or mitochondrial DNA sequences with high spatial resolution was identified as likely to provide further benefit.

The instant disclosure provides methods and compositions that allow for unbiased capture of genomic and mitochondrial DNA from tissue in a high-throughput manner (i.e. read-outs are obtained via next-generation sequencing) and with high (up to 10 μm) spatial resolution.

Two key concepts that distinguish the instant methods and compositions from many prior approaches are: 1) Spatial resolution can be set up by manufacture of a dense monolayer of polystyrene beads with “known locations”: each bead (approx. 10 μm diameter) is coated with oligonucleotides that contain a unique spatial barcode whose sequence is read out through imaging (as described further infra, this component of the instant disclosure is noted as shared with the “Slide-seq” approach of PCT/US19/30194); and 2) High-throughput sequencing of cellular DNA can retain spatial context for the cellular DNA within the tissue if the DNA is processed in situ such that DNA fragments can be linked locally to the unique spatial barcode from the nearest bead(s). In certain aspects of the instant disclosure, the tissue section is treated with a permeabilizing agent and/or a DNA fragmenting agent, and is then contacted with the dense monolayer of oligonucleotide-coated beads that contain a unique spatial barcode whose sequence is read out through imaging (prior to tissue contact).

The “Slide-seq” approach of PCT/US19/30194 successfully demonstrated that a bead monolayer with detectable/known spatial barcodes could preserve spatial information of macromolecules, particularly of transcriptomes, in a tissue. The instant disclosure builds upon the “Slide-seq” approach to provide enhanced access to cellular DNA, specifically by processing tissue sections in a manner that allows for simultaneous readout of cellular DNA sequences and location of such DNA sequences within the tissue by next-generation sequencing (i.e. without imaging the tissue on a microscope). The approaches and compositions of the instant disclosure therefore provide for improved assessment of the following, among other advantages:

1) Improved, high-resolution spatial assessment of copy number variation (CNV). CNV analysis has proven powerful in the past in tracing tumor lineages. However, since CNV assessment of tumor lineages has previously required dissociation of the tissue, it has mostly not been possible to address the spatial relationship between and within lineages. The instant disclosure empowers such studies by capturing genomic DNA in situ from tumor tissues, identifying changes of CNVs to determine lineages, and using the spatial barcodes that are associated with each DNA fragment to probe for any spatial patterns of cancer lineages. Exemplary forms of CNV that can be detected with the methods and compositions of the instant disclosure include trisomy and other chromosomal and/or sub-chromosomal amplification events, and regions of aneuploidy, among others.

2) Mitochondrial lineage tracing. Somatic mutations in mitochondrial DNA have been recently shown to enable lineage inference in hematopoietic cells and solid tumors. Since the instant approach is capable of capturing all forms of cellular DNA, including mitochondrial DNA, it can be applied to study clonal dynamics and their spatial relationship in tissues.

3) High-resolution spatial assessment of epigenetic regulation, including, e.g., high-resolution spatial assessment of DNA methylation patterns. The previously described Assay for Transposase-Accessible Chromatin using sequencing (ATAC-seq) has led to numerous new insights into cell state transitions and gene regulation, but has so far not been able to preserve spatial information with high resolution. Using the approach of the instant disclosure DNA libraries have now been successfully prepared that show a significant enrichment for transcription start sites, which indicates that the instant approach can specifically capture accessible chromatin. The instant disclosure can therefore be used to study how different cell types with varying epigenetic states are spatially distributed within a tissue.

4) Improved identification of regions of monoallelic gene expression and gene dosage in an assayed tissue. A variety of genes are expressed from one allele only (e.g. through imprinting, X chromosome inactivation, or autosomal random monoallelic expression) but many aspects of their regulation, such as tissue-specific skewing towards one allele or the stability of repression of autosomal alleles, are poorly understood. In certain aspects, the instant disclosure allows for capture specifically of accessible chromatin (i.e. active alleles), which can therefore be used to study the spatial distribution of mono- vs. bi-allelic gene expression.

5) High-resolution, spatial evaluation of gene therapy deliverables to tissue, including, e.g., identification of cellular delivery of CRISPR/Cas9 plasmid(s) and/or gels, TALEN plasmid(s) and/or gels, viral vectors (e.g., AAV), expression vectors/plasmids in general, etc.

6) Assessment of synthetic DNA arrays for sequence-specific quantities and distributions of DNA upon the synthetic DNA array. Certain aspects of the approach disclosed in PCT/US19/30194 describe capture of RNA, in certain embodiments by including a poly-dT stretch on a bead-attached oligonucleotide. It is herein specifically contemplated that DNA from synthetic arrays can be allowed to hybridize to a sequenced puck, using known sequences attached to the DNA (so that target DNA can be captured by reverse-complement sequences included in the bead-attached oligonucleotides. Tagmentation as described herein is an approach that allows for addition of such adapter sequences.

Certain aspects of the instant disclosure expand upon the original “Slide-seq” technology platform of PCT/US19/30194, specifically employing the same beads, arrays and sequencing chemistry as “Slide-seq”.

Additional details of the instant disclosure are provided in the following sections.

### Permeabilizing Agents

Certain aspects of the instant disclosure feature permeabilizing agents, examples of which tend to compromise and/or remove the protective boundary of lipids often surrounding cellular macromolecules. Disruption of cellular lipid barriers via administration of a permeabilizing agent can provide enhanced physical access to cellular macromolecules, such as DNA, that might otherwise be relatively inaccessible. Specifically contemplated examples of permeabilizing agents include, without limitation: Triton X-100, NP-40, methanol, acetone, Tween 20, saponin, Leucoperm™, and digitonin, among others.

### DNA Fragmenting Agents

Some aspects of the instant disclosure employ DNA fragmenting agents, which typically allow for capture of target DNA molecules and performance of high throughput DNA sequencing upon such captured target DNA molecules (e.g., target DNA of accessible chromatin in situ). In certain embodiments for DNA preparation, a hyperactive variant of the Tn5 transposase that mediates the fragmentation of double-stranded DNA and ligates synthetic oligonucleotides at both ends can be employed. In the Tn5 tagmentation reaction, the Tn5 enzyme randomly cleaves accessible double stranded DNA into fragments and tags the DNA, by end-joining to the 5′-ends synthetic “mosaic ends,” or adapter sequences. In embodiments of the instant disclosure, the mosaic ends, or Tn5 adapter sequences, complement bead adapter and Illumina® Handle sequences.

A hyperactive variant of the Tn5 transposase employed herein in the below Examples was derived from the naturally occurring wild-type Tn5 transposase. Three missense mutations in the 476 residues of the Tn5 protein have typically been introduced: E54K, M56A, L372P, to produce the hyperactive variant. The wild type Tn5 transposon also contains two near-identical insertion sequences (IS50L and IS50R) flanking three antibiotic resistance genes (Reznikoff 2008). Each IS50 contains two inverted 19-base pair end sequences. However, because wild-type end sequences exhibited relatively low activity, they were replaced in vitro by synthetic hyperactive mosaic end sequences. A complex of the hyperactive Tn5 transposase with the 19-base pair mosaic end sequences therefore can provoke the mutant Tn5 to induce DNA fragmentation.

Specific forms of DNA fragmenting agents/enzymes for use in the instant disclosure include, without limitation: transposases (including non-Tn5 transposases), DFF40, and DNases, including restriction endonucleases. In other embodiments of the instant disclosure, H₂O₂ (hydrogen peroxide) can be employed to fragment DNA.

### Tagmentation

In certain aspects, the instant disclosure employs a Tn5 tagmentation reaction, in which a Tn5 enzyme randomly cleaves double stranded DNA into fragments and tags the DNA, by end-joining to the 5′-ends, synthetic “mosaic ends,” or adapter sequences. In one embodiment of the instant disclosure, the mosaic ends, or Tn5 adapter sequences, complement bead adapter and Illumina® Handle sequences. In embodiments, “tagmentation” refers to sequencing techniques that employ a hyperactive mutant form of Tn5 as described above. The tagged DNA fragments can then be purified, PCR-amplified, and sequenced using next-generation sequencing. Sequencing reads can then be used to infer regions of increased accessibility as well as to map regions of transcription factor binding sites and nucleosome positions. The number of reads for a region correlate with the degree of accessibility of the chromatin region.

### Nucleosome Disrupting Agents

In some embodiments of the instant disclosure, chromatin structure is disrupted to allow for greater access to chromatin regions that might otherwise be inaccessible/under-represented, thereby providing improved genomic representation of assayed DNA molecules in such regions. As exemplified herein, nucleosomes can be disrupted via contact with HCl, SDS and/or a protease/proteinase. Chromatin structure and/or nucleosomes can also be disrupted by a number of other agents and approaches, including, without limitation: naturally occurring Drosophila nucleosome remodeling proteins, which include but are not limited to: nucleosome remodeling factor (NURF), ATP-dependent chromatin assembly and remodeling factor (ACF) (Ito et al. 1997), and the chromatin accessibility complex (CHRAC). The common catalytic core of these remodeling factors is ISWI, a nucleosome-stimulated ATPase of the SWI2/SNF2 superfamily. In other embodiments, nucleosome disruption may be performed using ISWI-containing nucleosome remodeling complexes from yeast (ISW1-ISW2 complex; Tsukiyama et al. 1999) or humans (RSF; LeRoy et al. 1998). In other embodiments, the GAGA transcription factor may be used to disrupt nucleosomes (Okada et al. 1998).

### Solid Supports

In certain aspects, the present disclosure provides a method for generating and using a spatially tagged array of microbeads to perform deep DNA abundance assessment upon sectioned tissue samples, with high image resolution. The method can include the steps of (a) attaching different nucleic acid probes to beads that are then captured upon a solid support to produce randomly located probe-possessing beads on the solid support, wherein the different nucleic acid probes each includes a barcode sequence (that is shared by all such nucleic acid probes of a single bead), and wherein each of the randomly located beads includes a different barcode sequence(s) from other randomly located beads on the solid support; (b) performing a nucleic acid detection reaction on the solid support to determine the barcode sequences of the randomly located beads on the solid support; (c) contacting a biological specimen with the solid support that has the randomly located beads; (d) hybridizing the probes presented by the randomly located beads to target nucleic acids from portions of the biological specimen that are proximal to the randomly located beads; and (e) extending the probes of the randomly located beads to produce extended probes that include the barcode sequences and sequences from the target nucleic acids, thereby spatially tagging the nucleic acids of the biological specimen.

Any of a variety of solid supports can be used in a method, composition or apparatus of the present disclosure. Particularly useful solid supports are those used for nucleic acid arrays. Examples include glass, modified glass, functionalized glass, inorganic glasses, microspheres (e.g. inert and/or magnetic particles), plastics, polysaccharides, nylon, nitrocellulose, ceramics, resins, silica, silica-based materials, carbon, metals, an optical fiber or optical fiber bundles, polymers and multiwell (e.g. microtiter) plates. Exemplary plastics include acrylics, polystyrene, copolymers of styrene and other materials, polypropylene, polyethylene, polybutylene, polyurethanes and TeflonTM. Exemplary silica-based materials include silicon and various forms of modified silicon.

In particular embodiments, a solid support can be within or part of a vessel such as a well, tube, channel, cuvette, Petri plate, bottle or the like. Optionally, the vessel is a flow-cell, for example, as described in WO 2014/142841 Al; U.S. Pat. App. Pub. No. 2010/0111768 A1 and U.S. Pat. No. 8,951,781 or Bentley et al., Nature 456:53-59 (2008), each of which is incorporated herein by reference. Exemplary flow-cells are those that are commercially available from lllumina, Inc. (San Diego, Calif.) for use with a sequencing platform such as a Genome Analyzer®, MiSeq®, NextSeq® or HiSeq® platform. Optionally, the vessel is a well in a multiwell plate or microtiter plate.

In certain embodiments, a solid support can include a gel coating. Attachment, e.g., of nucleic acids to a solid support via a gel is exemplified by flow cells available commercially from lllumina Inc. (San Diego, Calif.) or described in US Pat. App. Pub. Nos. 2011/0059865 A1, 2014/0079923 A1, or 2015/0005447 A1; or PCT Publ. No. WO 2008/093098, each of which is incorporated herein by reference. Exemplary gels that can be used in the methods and apparatus set forth herein include, but are not limited to, those having a colloidal structure, such as agarose; polymer mesh structure, such as gelatin; or cross-linked polymer structure, such as polyacrylamide, SFA (see, for example, US Pat. App. Pub. No. 2011/0059865 A1, which is incorporated herein by reference) or PAZAM (see, for example, US Pat. App. Publ. Nos. 2014/0079923 A1, or 2015/0005447 A1, each of which is incorporated herein by reference).

In some embodiments, a solid support can be configured as an array of features to which beads can be attached. The features can be present in any of a variety of desired formats. For example, the features can be wells, pits, channels, ridges, raised regions, pegs, posts or the like. Exemplary features include wells that are present in substrates used for commercial sequencing platforms sold by 454 LifeSciences (a subsidiary of Roche, Basel Switzerland) or Ion Torrent (a subsidiary of Life Technologies, Carlsbad California). Other substrates having wells include, for example, etched fiber optics and other substrates described in U.S. Pat Nos. 6,266,459; 6,355,431; 6,770,441 ; 6,859,570; 6,210,891 ; 6,258,568; 6,274,320; US Pat app. Publ. Nos. 2009/0026082 A1; 2009/0127589 A1; 2010/0137143 A1; 2010/0282617 A1 or PCT Publication No. WO 00/63437, each of which is incorporated herein by reference. In some embodiments, wells of a substrate can include gel material (with or without beads) as set forth in US Pat. App. Publ. No. 2014/0243224 A1, which is incorporated herein by reference.

Features can appear on a solid support as a grid of spots or patches. The features can be located in a repeating pattern or in an irregular, non-repeating pattern. Optionally, repeating patterns can include hexagonal patterns, rectilinear patterns, grid patterns, patterns having reflective symmetry, patterns having rotational symmetry, or the like. Asymmetric patterns can also be useful. The pitch of an array can be the same between different pairs of nearest neighbor features or the pitch can vary between different pairs of nearest neighbor features.

In particular embodiments, features on a solid support can each have an area that is larger than about 100 nm², 250 nm², 500 nm², 1 μm², 2.5 μm², 5 μm², 10 μm² or 50 μm². Alternatively or additionally, features can each have an area that is smaller than about 50 μm², 25 μm², 10 μm², 5 μm², 1 μm², 500 nm², or 100 nm². The preceding ranges can describe the apparent area of a bead or other particle on a solid support when viewed or imaged from above.

### Beads

Certain aspects of the instant disclosure employ a collection of beads or other particles, to which oligonucleotides are attached. Suitable bead compositions include those used in peptide, nucleic acid and organic moiety synthesis, including, but not limited to, plastics, ceramics, glass, polystyrene, methylstyrene, acrylic polymers, paramagnetic materials, thoriasol, carbon graphite, titanium dioxide, latex or cross-linked dextrans such as Sepharose, cellulose, nylon, cross-linked micelles and Teflon may all be used. “Microsphere Detection Guide” from Bangs Laboratories, Fishers IN is a helpful guide, which is incorporated herein by reference in its entirety. The beads need not be spherical; irregular particles may be used. In addition, the beads may be porous, thus increasing the surface area of the bead available for either capture probe attachment or tag attachment. The bead sizes can range from nanometers, for example, 100 nm, to millimeters, for example, 1 mm, with beads from about 0.2 μm to about 200 μm commonly employed, and from about 5 to about 20 μm being within the range currently exemplified, although in some embodiments smaller or larger beads may be used.

The particles can be suspended in a solution or they can be located on the surface of a substrate (e.g., arrayed upon the surface of a solid support, such as a glass slide). Art-recognized examples of arrays having beads located on a surface include those wherein beads are located in wells such as a BeadChip array (lllumina Inc., San Diego Calif.), substrates used in sequencing platforms from 454 LifeSciences (a subsidiary of Roche, Basel Switzerland) or substrates used in sequencing platforms from Ion Torrent (a subsidiary of Life Technologies, Carlsbad Calif.). Other solid supports having beads located on a surface are described in U.S. Pat. Nos. 6,266,459; 6,355,431; 6,770,441; 6,859,570; 6,210,891; 6,258,568; or 6,274,320; US Pat. App. Publ. Nos. 2009/0026082 A12009/0127589 A1; 2010/0137143 A1; or 2010/0282617 A1 or PCT Publication No. WO 00/63437, each of which is incorporated herein by reference. Several of the above references describe methods for attaching nucleic acid probes to beads prior to loading the beads in or on a solid support. As such, the collection of beads can include different beads each having a unique (or sufficiently unique and/or near-unique, as described elsewhere herein) probe attached. It will however, be understood that the beads can be made to include universal primers, and the beads can then be loaded onto an array, thereby forming universal arrays for use in a method set forth herein. The solid supports typically used for bead arrays can be used without beads. For example, nucleic acids, such as probes or primers can be attached directly to the wells or to gel material in wells. Thus, the above references are illustrative of materials, compositions or apparatus that can be modified for use in the methods and compositions set forth herein.

Accordingly, the instant methods can employ an array of beads, wherein different nucleic acid probes are attached to different beads in the array. In this embodiment, each bead can be attached to a different nucleic acid probe and the beads can be randomly distributed on the solid support in order to effectively attach the different nucleic acid probes to the solid support. Optionally, the solid support can include wells having dimensions that accommodate no more than a single bead. In such a configuration, the beads may be attached to the wells due to forces resulting from the fit of the beads in the wells. As described elsewhere herein, it is also possible to use attachment chemistries or capture materials (e.g., liquid electrical tape) to adhere or otherwise stably associate the beads with a solid support, optionally including holding the beads in wells that may or may not be present on a solid support.

Nucleic acid probes that are attached to beads can include barcode sequences. A population of the beads can be configured such that each bead is attached to only one type of barcode (e.g., a spatial barcode) and many different beads each with a different barcode are present in the population. In this embodiment, randomly distributing the beads to a solid support will result in randomly locating the nucleic acid probe-presenting beads (and their respective barcode sequences) on the solid support. In some cases, there can be multiple beads with the same barcode sequence such that there is redundancy in the population. However, randomly distributing a redundancy-comprising population of beads on a solid support—especially one that has a capacity that is greater than the number of unique barcodes in the bead population—will tend to result in redundancy of barcodes on the solid support, which will tend to reduce image resolution in the context of the instant disclosure (i.e., where the precise location of a barcoded bead cannot be resolved due to redundancy of barcode use within an arrayed population of beads, it is contemplated that such redundant locations will simply be eliminated from an ultimate image produced by methods of the instant disclosure, or other modes of adjustment (e.g., normalization and/or averaging of values) may also be employed to address such redundancies). Alternatively, in preferred embodiments, the number of different barcodes in a population of beads can exceed the capacity of the solid support in order to produce an array that is not redundant with respect to the population of barcodes on the solid support. The capacity of the solid support will be determined in some embodiments by the number of features (e.g. single-bead occupancy wells) that attach or otherwise accommodate a bead.

A bead or other nucleic acid-presenting solid support of the instant disclosure can include, or can be made by the methods set forth herein to attach, a plurality of different nucleic acid probes. For example, a bead or other nucleic acid-presenting solid support can include at least 10, 100, 1×10³, 1×10⁴, 1×10⁵, 1×10⁶, 1×10⁷, 1×10⁸, 1×10⁹ or more different probes. Alternatively or additionally, a bead or other nucleic acid-presenting solid support can include at most 1×10⁹, 1×10⁸, 1×10⁷, 1×10⁶, 1×10⁵, 1×10⁴, 1×10³, 100, or fewer different probes. It will be understood that each of the different probes can be present in several copies, for example, when the probes have been amplified to form a cluster. Thus, the above ranges can describe the number of different nucleic acid clusters on a bead or other nucleic acid-presenting solid support of the instant disclosure. It will also be understood that the above ranges can describe the number of different barcodes, target capture sequences, or other sequence elements set forth herein as being unique (or sufficiently unique) to particular nucleic acid probes. Alternatively or additionally, the ranges can describe the number of extended probes or modified probes created on a bead or other nucleic acid-presenting solid support of the instant disclosure using a method set forth herein.

Features may be present on a bead or other solid support of the instant disclosure prior to contacting the bead or other solid support with nucleic acid probes. For example, in embodiments where probes are attached to a bead or other solid support via hybridization to primers, the primers can be attached at the features, whereas interstitial areas outside of the features substantially lack any of the primers. Nucleic acid probes can be captured at preformed features on a bead or other solid support, and optionally amplified on the bead or other solid support, e.g., using methods set forth in U.S. Pat. Nos. 8,895,249 and 8,778,849 and/or U.S. Patent Publication No. 2014/0243224 A1, each of which is incorporated herein by reference. Alternatively, a bead or other solid support may have a lawn of primers or may otherwise lack features. In this case, a feature can be formed by virtue of attachment of a nucleic acid probe on the bead or other solid support. Optionally, the captured nucleic acid probe can be amplified on the bead or other solid support such that the resulting cluster becomes a feature. Although attachment is exemplified above as capture between a primer and a complementary portion of a probe, it will be understood that capture moieties other than primers can be present at pre-formed features or as a lawn. Other exemplary capture moieties include, but are not limited to, chemical moieties capable of reacting with a nucleic acid probe to create a covalent bond or receptors capable of binding non-covalently to a ligand on a nucleic acid probe.

A step of attaching nucleic acid probes to a bead or other solid support can be carried out by providing a fluid that contains a mixture of different nucleic acid probes and contacting this fluidic mixture with the bead or other solid support. The contact can result in the fluidic mixture being in contact with a surface to which many different nucleic acid probes from the fluidic mixture will attach. Thus, the probes have random access to the surface (whether the surface has pre-formed features configured to attach the probes or a uniform surface configured for attachment). Accordingly, the probes can be randomly located on the bead or other solid support.

The total number and variety of different probes that end up attached to a surface can be selected for a particular application or use. For example, in embodiments where a fluidic mixture of different nucleic acid probes is contacted with a bead or other solid support for purposes of attaching the probes to the support, the number of different probe species can exceed the occupancy of the bead or other solid support for probes. Thus, the number and variety of different probes that attach to the bead or other solid support can be equivalent to the probe occupancy of the bead or other solid support.

Alternatively, the number and variety of different probe species on the bead or other solid support can be less than the occupancy (i.e. there will be redundancy of probe species such that the bead or other solid support may contain multiple features having the same probe species). Such redundancy can be achieved, for example, by contacting the bead or other solid support with a fluidic mixture that contains a number and variety of probe species that is substantially lower than the probe occupancy of the bead or other solid support.

Attachment of the nucleic acid probes can be mediated by hybridization of the nucleic acid probes to complementary primers that are attached to the bead or other solid support, chemical bond formation between a reactive moiety on the nucleic acid probe and the bead or other solid support (examples are set forth in U.S. Pat. Nos. 8,895,249 and 8,778,849, and in U.S. Patent Publication No. 2014/0243224 A1, each of which is incorporated herein by reference), affinity interactions of a moiety on the nucleic acid probe with a bead- or other solid support-bound moiety (e.g. between known receptor-ligand pairs such as streptavidin-biotin, antibody-epitope, lectin-carbohydrate and the like), physical interactions of the nucleic acid probes with the bead or other solid support (e.g. hydrogen bonding, ionic forces, van der Waals forces and the like), or other interactions known in the art to attach nucleic acids to surfaces.

In some embodiments, attachment of a nucleic acid probe is non-specific with regard to any sequence differences between the nucleic acid probe and other nucleic acid probes that are or will be attached to the bead or other solid support. For example, different probes can have a universal sequence that complements surface-attached primers or the different probes can have a common moiety that mediates attachment to the surface. Alternatively, each of the different probes (or a subpopulation of different probes) can have a unique (or sufficiently unique) sequence that complements a unique (or sufficiently unique) primer on the bead or other solid support or they can have a unique (or sufficiently unique) moiety that interacts with one or more different reactive moiety on the bead or other solid support. In such cases, the unique (or sufficiently unique) primers or unique (or sufficiently unique) moieties can, optionally, be attached at predefined locations in order to selectively capture particular probes, or particular types of probes, at the respective predefined locations.

One or more features on a bead or other solid support can each include a single molecule of a particular probe. The features can be configured, in some embodiments, to accommodate no more than a single nucleic acid probe molecule. However, whether or not the feature can accommodate more than one nucleic acid probe molecule, the feature may nonetheless include no more than a single nucleic acid probe molecule. Alternatively, an individual feature can include a plurality of nucleic acid probe molecules, for example, an ensemble of nucleic acid probe molecules having the same sequence as each other. In particular embodiments, the ensemble can be produced by amplification from a single nucleic acid probe template to produce amplicons, for example, as a cluster attached to the surface.

A method as set forth herein can employ any of a variety of amplification techniques. Exemplary amplification techniques that can be used include, but are not limited to, polymerase chain reaction (PCR), rolling circle amplification (RCA), multiple displacement amplification (MDA), and random prime amplification (RPA). In some embodiments the amplification can be carried out in solution, for example, when features of an array are capable of containing amplicons in a volume having a desired capacity. In certain embodiments, an amplification technique used in a method of the present disclosure will be carried out on solid phase. For example, one or more primer species (e.g. universal primers for one or more universal primer binding site present in a nucleic acid probe) can be attached to a bead or other solid support. In PCR embodiments, one or both of the primers used for amplification can be attached to a bead or other solid support (e.g. via a gel). Formats that utilize two species of primers attached to a bead or other solid support are often referred to as bridge amplification because double stranded amplicons form a bridge-like structure between the two surface-attached primers that flank the template sequence that has been copied. Exemplary reagents and conditions that can be used for bridge amplification are described, for example, in U.S. Pat. Nos. 5,641,658; 7,115,400; and 8,895,249; and/or U.S. Patent Publication Nos. 2002/0055100 A1, 2004/0096853 A1, 2004/0002090 A1, 2007/0128624 A1 and 2008/0009420 A1, each of which is incorporated herein by reference. Solid-phase PCR amplification can also be carried out with one of the amplification primers attached to a bead or other solid support and the second primer in solution. An exemplary format that uses a combination of a surface-attached primer and soluble primer is the format used in emulsion PCR as described, for example, in Dressman et al., Proc. Natl. Acad. Sci. USA 100:8817-8822 (2003), WO 05/010145, or U.S. Patent Publication Nos. 2005/0130173 or 2005/0064460, each of which is incorporated herein by reference. Emulsion PCR is illustrative of the format and it will be understood that for purposes of the methods set forth herein the use of an emulsion is optional and indeed for several embodiments an emulsion is not used.

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and U.S. Patent Publication No. 2007/0099208, each of which is incorporated herein by reference. Primers used for RCA can be in solution or attached to a bead or other solid support. The primers can be one or more of the universal primers described herein.

MDA techniques can be modified for use in a method of the present disclosure. Some basic principles and useful conditions for MDA are described, for example, in Dean et al., Proc Natl. Acad. Sci. USA 99:5261 -66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference. Primers used for MDA can be in solution or attached to a bead or other solid support at an amplification site. Again, the primers can be one or more of the universal primers described herein.

In particular embodiments a combination of the above-exemplified amplification techniques can be used. For example, RCA and MDA can be used in a combination wherein RCA is used to generate a concatameric amplicon in solution (e.g. using solution-phase primers). The amplicon can then be used as a template for MDA using primers that are attached to a bead or other solid support (e.g. universal primers). In this example, amplicons produced after the combined RCA and MDA steps will be attached to the bead or other solid support.

Nucleic acid probes that are used in a method set forth herein or present in an apparatus or composition of the present disclosure can include barcode sequences, and for embodiments that include a plurality of different nucleic acid probes, each of the probes can include a different barcode sequence from other probes in the plurality. Barcode sequences can be any of a variety of lengths.

Longer sequences can generally accommodate a larger number and variety of barcodes for a population. Generally, all probes in a plurality will have the same length barcode (albeit with different sequences), but it is also possible to use different length barcodes for different probes. A barcode sequence can be at least 2, 4, 6, 8, 10, 12, 15, 20 or more nucleotides in length. Alternatively or additionally, the length of the barcode sequence can be at most 20, 15, 12, 10, 8, 6, 4 or fewer nucleotides. Examples of barcode sequences that can be used are set forth, for example, in U.S. Patent Publication No. 2014/0342921 and U.S. Pat. No. 8,460,865, each of which is incorporated herein by reference.

A method of the present disclosure can include a step of performing a nucleic acid detection reaction on a bead or other solid support to determine barcode sequences of nucleic acid probes that are located on the bead or other solid support. In many embodiments the probes are randomly located on the bead or other solid support and the nucleic acid detection reaction provides information to locate each of the different probes. Exemplary nucleic acid detection methods include, but are not limited to, nucleic acid sequencing of a probe, hybridization of nucleic acids to a probe, ligation of nucleic acids that are hybridized to a probe, extension of nucleic acids that are hybridized to a probe, extension of a first nucleic acid that is hybridized to a probe followed by ligation of the extended nucleic acid to a second nucleic acid that is hybridized to the probe, or other methods known in the art such as those set forth in U.S. Pat. No. 8,288,103 or 8,486,625, each of which is incorporated herein by reference.

Sequencing techniques, such as sequencing-by-synthesis (SBS) techniques, are a useful method for determining barcode sequences. SBS can be carried out as follows. To initiate a first SBS cycle, one or more labeled nucleotides, DNA polymerase, SBS primers etc., can be contacted with one or more features on a bead or other solid support (e.g. feature(s) where nucleic acid probes are attached to the bead or other solid support). Those features where SBS primer extension causes a labeled nucleotide to be incorporated can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the bead or other solid support (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and U.S. Patent Publication No. 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 1 1 (1), 3-1 1 (2001); Ronaghi et al. Science 281 (5375), 363 (1998); or U.S. Pat. Nos. 6,210,891, 6,258,568 or 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system.

Excitation radiation sources used for fluorescence based detection systems are not necessary for pyrosequencing procedures. Useful fluidic systems, detectors and procedures that can be used for application of pyrosequencing to apparatus, compositions or methods of the present disclosure are described, for example, in PCT Patent Publication No. W02012/058096, US Patent Publication No. 2005/0191698 A1, or U.S. Pat. Nos. 7,595,883 or 7,244,559, each of which is incorporated herein by reference.

Sequencing-by-ligation reactions are also useful including, for example, those described in Shendure et al. Science 309:1728-1732 (2005); or US Pat. Nos. 5,599,675 or 5,750,341, each of which is incorporated herein by reference. Some embodiments can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251 (4995), 767-773 (1995); or PCT Publication No. WO 1989/10977, each of which is incorporated herein by reference. In both sequencing-by-ligation and sequencing-by-hybridization procedures, target nucleic acids (or amplicons thereof) that are present at sites of an array are subjected to repeated cycles of oligonucleotide delivery and detection. Compositions, apparatus or methods set forth herein or in references cited herein can be readily adapted for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some sequencing embodiments can utilize methods involving the real-time monitoring of DNA polymerase activity. For example, nucleotide incorporations can be detected through fluorescence resonance energy transfer (FRET) interactions between a fluorophore-bearing polymerase and y-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); and Korlach et al. Proc. Natl. Acad. Sci. USA 105, 1 176-1 181 (2008), each of which is incorporated herein by reference.

Some sequencing embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies and Thermo Fisher subsidiary) or sequencing methods and systems described in U.S. Patent Publication Nos. 2009/0026082 A1; 2009/0127589 A1; 2010/0137143 A1; or U.S. Publication No. 2010/0282617 A1, each of which is incorporated herein by reference.

Nucleic acid hybridization techniques are also useful methods for determining barcode sequences. In some cases combinatorial hybridization methods can be used such as those used for decoding of multiplex bead arrays (see, e.g., U.S. Pat. No. 8,460,865, which is incorporated herein by reference). Such methods utilize labelled nucleic acid decoder probes that are complementary to at least a portion of a barcode sequence. A hybridization reaction can be carried out using decoder probes having known labels such that the location where the labels end up on the bead or other solid support identifies the nucleic acid probes according to rules of nucleic acid complementarity. In some cases, pools of many different probes with distinguishable labels are used, thereby allowing a multiplex decoding operation. The number of different barcodes determined in a decoding operation can exceed the number of labels used for the decoding operation. For example, decoding can be carried out in several stages where each stage constitutes hybridization with a different pool of decoder probes. The same decoder probes can be present in different pools but the label that is present on each decoder probe can differ from pool to pool (i.e. each decoder probe is in a different “state” when in different pools).

Various combinations of these states and stages can be used to expand the number of barcodes that can be decoded well beyond the number of distinct labels available for decoding. Such combinatorial methods are set forth in further detail in U.S. Pat. No. 8,460,865 or Gunderson et al., Genome Research 14:870-877 (2004), each of which is incorporated herein by reference.

A method of the present disclosure can include a step of contacting a biological specimen (i.e., a sectioned tissue sample treated with a permeabilizing agent and/or a DNA fragmenting agent and/or an agent for disrupting nucleosomes/histones or other cellular components that confer genomic/chromatin structure, thereby allowing for increased genomic accessibility while using the instant approach) with a bead or other solid support that has nucleic acid probes attached thereto. In some embodiments, the nucleic acid probes are randomly located on the bead or other solid support. The identity and location of the nucleic acid probes may have been decoded prior to contacting the biological specimen with the bead or other solid support.

Alternatively, the identity and location of the nucleic acid probes can be determined after contacting the bead or other solid support with the biological specimen.

### Bead-Attached Oligonucleotides

Certain aspects of the instant disclosure employ a nucleotide- or oligonucleotide-adorned bead, where the bead-attached oligonucleotide includes one or more of the following: a linker; an identical sequence for use as a sequencing priming site; a uniform or near-uniform nucleotide or oligonucleotide sequence; optionally, a Unique Molecular Identifier (UMI) which differs for each priming site can be included; an oligonucleotide redundant sequence for capturing DNA; and at least one oligonucleotide barcode which provides a substrate for spatial identification of an individual bead's position within a bead array. Exemplified bead-attached oligonucleotides of the instant disclosure include an oligonucleotide spatial barcode designed to be unique to each bead within a bead array (or at least wherein the majority of such barcodes are unique to a bead within a bead array—e.g., it is expressly contemplated here and elsewhere herein that a bead array possessing only a small fraction of beads (e.g., even up to 10%, 20%, 30% or 40% or more of total beads) having non-unique spatial barcodes (e.g., attributable to a relative lack of degeneracy within the bead population, e.g., due to a probabilistically determinable lack of sequence degeneracy calculated as possible within the bead population, as then compared to the number of sites across which the bead population is ultimately distributed and/or due to an artifact such as non-randomness of bead association occurring during pool-and-split rounds of oligonucleotide synthesis, etc.) could still yield high resolution genome identity images, even while removing (or otherwise adjusting for) any beads that turn out to be redundant in barcode within the array). This spatial barcode provides a substrate for identification.

It is contemplated that certain bead-attached oligonucleotides of the instant disclosure can also include a linker (optionally a cleavable linker); a Unique Molecular Identifier (UMI) which differs for each priming site (as described below and as known in the art, e.g., see WO 2016/040476); a spatial barcode as described above and elsewhere herein; and a common sequence (“PCR handle”) to enable PCR amplification after cellular DNAs are attached to microparticles.

It is expressly contemplated that oligonucleotide sequences designed for capture of a broad range of DNA molecules as described here and elsewhere herein, can be used. In particular, target DNA sequence-specific oligonucleotide-directed capture of DNA molecules is contemplated for the bead-attached oligonucleotides of the instant disclosure; for instance, a target gene-specific capture sequence can be incorporated into oligonucleotide sequences (e.g., for purpose of capturing a select range of cellular DNA molecules).

Exemplary split-and-pool synthesis of the bead barcode: To generate the cell barcode, the pool of microparticles (here, microbeads) is repeatedly split into four equally sized oligonucleotide synthesis reactions, to which one of the four DNA bases is added, and then pooled together after each cycle, in a total of 12 split-pool cycles. The barcode synthesized on any individual bead reflects that bead's unique (or sufficiently unique) path through the series of synthesis reactions. The result is a pool of microparticles, each possessing one of 4¹² (16,777,216) possible sequences on its entire complement of primers. Extension of the split-pool process can provide for, e.g., production of an even greater number of possible spatial barcode sequences for use in the compositions and methods of the instant disclosure. However, as noted above, functional use of spatial barcodes does not require complete non-redundancy of spatial barcodes among all beads of a bead array. Rather, provided that the majority of such barcodes are unique to a bead within a bead array, it is expressly contemplated that a bead array possessing only a small fraction of beads (e.g., even up to 10%, 20%, 30% or 40% or more of total beads) having non-unique spatial barcodes (e.g., attributable to an artifact such as non-randomness of bead association having occurred during pool-and-split rounds of oligonucleotide synthesis, or simply to the likelihood that an array of a million beads derived from a ten million-fold complex library would still be expected to include a number of beads having redundant spatial barcodes in pairwise comparisons) could still yield high resolution DNA abundance images, where removal or other adjustment (averaging or other such adjustment) of any beads that turn out to be redundant in barcode within the array could be simply performed, e.g., during in silico spatial location assignment and/or image generation.

Exemplary synthesis of a unique molecular identifier (UMI). Following the completion of the “split-and-pool” synthesis cycles described above for generation of spatial barcodes, all microparticles are together subjected to eight rounds of degenerate synthesis with all four DNA bases available during each cycle, such that each individual primer receives one of 4⁸ (65,536) possible sequences (UMIs). A UMI is thereby provided that allows distinguishing between, e.g., individual bead-attached oligonucleotides upon the same bead which otherwise share a common spatial barcode (being that such oligonucleotides are attached to the same bead and therefore receive the same spatial barcode).

In some embodiments of the instant disclosure, the linker of a bead-attached oligonucleotide is a chemically-cleavable, straight-chain polymer. Optionally, the linker is photolabile, optionally a substituted hydrocarbon polymer. In certain embodiments, the linker of a bead-attached oligonucleotide is a non-cleavable, straight-chain polymer. Optionally, the linker is a non-cleavable, optionally substituted hydrocarbon polymer. In certain embodiments, the linker is a polyethylene glycol. In one embodiment, the linker is a PEG-C3 to PEG-24.

A nucleic acid probe used in a composition or method set forth herein can include a target capture moiety. In particular embodiments, the target capture moiety is a target capture sequence. The target capture sequence is generally complementary to a target sequence such that target capture occurs by formation of a probe-target hybrid complex. A target capture sequence can be any of a variety of lengths including, for example, lengths exemplified above in the context of barcode sequences.

In certain embodiments, a plurality of different nucleic acid probes can include different target capture sequences that hybridize to different target nucleic acid sequences from a biological specimen. Different target capture sequences can be used to selectively bind to one or more desired target nucleic acids from a biological specimen. In some cases, the different nucleic acid probes can include a target capture sequence that is common to all or a subset of the probes on a solid support. For example, the nucleic acid probes on a solid support can be complementary to a transposon adapter sequence, e.g., where tagmentation is performed. Such probes or amplicons thereof can hybridize to tagmented DNA molecules or amplicons thereof. Although target DNA insert sequences will differ, capture will be mediated by the common sequence regions complementary to transposon adapter sequences.

A method set forth herein can include a step of hybridizing nucleic acid probes, that are on a supported bead array, to target nucleic acids that are from portions of the biological specimen that are proximal to the probes. Generally, a target nucleic acid will flow or diffuse from a region of the biological specimen to an area of the probe-presenting bead array that is in proximity with that region of the specimen. Here the target nucleic acid will interact with nucleic acid probes that are proximal to the region of the specimen from which the target nucleic acid was released. A target-probe hybrid complex can form where the target nucleic acid encounters a complementary target capture sequence on a nucleic acid probe. The location of the target-probe hybrid complex will generally correlate with the region of the biological specimen from where the target nucleic acid was derived. In certain embodiments, the beads will include a plurality of nucleic acid probes, the biological specimen will release a plurality of target nucleic acids and a plurality of target-probe hybrids will be formed on the beads. The sequences of the target nucleic acids and their locations on the bead array will provide spatial information about the nucleic acid content of the biological specimen. Although the example above is described in the context of target nucleic acids that are released from a biological specimen, it will be understood that the target nucleic acids need not be released. Rather, the target nucleic acids may remain in contact with the biological specimen, for example, when they are attached to an exposed surface of the biological specimen in a way that the target nucleic acids can also bind to appropriate nucleic acid probes on the beads.

A method of the present disclosure can include a step of extending bead-attached probes to which target nucleic acids are hybridized. In embodiments where the probes include barcode sequences, the resulting extended probes will include the barcode sequences and sequences from the target nucleic acids (albeit in complementary form). The extended probes are thus spatially tagged versions of the target nucleic acids from the biological specimen. The sequences of the extended probes identify what nucleic acids are in the biological specimen and where in the biological specimen the target nucleic acids are located. It will be understood that other sequence elements that are present in the nucleic acid probes can also be included in the extended probes (see, e.g., description as provided elsewhere herein). Such elements include, for example, primer binding sites, cleavage sites, other tag sequences (e.g. sample identification tags), capture sequences, recognition sites for nucleic acid binding proteins or nucleic acid enzymes, or the like.

Extension of probes can be carried out using methods exemplified herein or otherwise known in the art for amplification of nucleic acids or sequencing of nucleic acids. In particular embodiments one or more nucleotides can be added to the 3′ end of a nucleic acid, for example, via polymerase catalysis (e.g. DNA polymerase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended. Exemplary methods for extending nucleic acids are set forth in US Pat. App. Publ. No. US 2005/0037393 or U.S. Pat. Nos. 8,288,103 or 8,486,625, each of which is incorporated herein by reference.

All or part of a target nucleic acid that is hybridized to a nucleic acid probe can be copied by extension. For example, an extended probe can include at least, 1, 2, 5, 10, 25, 50, 100, 200, 500, 1000 or more nucleotides that are copied from a target nucleic acid. The length of the extension product can be controlled, for example, using reversibly terminated nucleotides in the extension reaction and running a limited number of extension cycles. The cycles can be run as exemplified for SBS techniques and the use of labeled nucleotides is not necessary.

Accordingly, an extended probe produced in a method set forth herein can include no more than 1000, 500, 200, 100, 50, 25, 10, 5, 2 or 1 nucleotides that are copied from a target nucleic acid. Of course extended probes can be any length within or outside of the ranges set forth above.

It will be understood that probes used in a method, composition or apparatus set forth herein need not be nucleic acids. Other molecules can be used such as proteins, carbohydrates, small molecules, particles or the like. Probes can be a combination of a nucleic acid component (e.g. having a barcode, primer binding site, cleavage site and/or other sequence element set forth herein) and another moiety (e.g. a moiety that captures or modifies a target nucleic acid).

A method set forth herein can further include a step of acquiring an image of a biological specimen that is in contact with a bead array. The solid support can be in any of a variety of states set forth herein. For example, the bead array can include attached nucleic acid probes or clusters derived from attached nucleic acid probes.

A method of the present disclosure can further include a step of removing one or more extended probes from a bead. In particular embodiments, the probes will have included a cleavage site such that the product of extending the probes will also include the cleavage site. Alternatively, a cleavage site can be introduced into a probe during a modification step. For example a cleavage site can be introduced into an extended probe during the extension step.

Exemplary cleavage sites include, but are not limited to, moieties that are susceptible to a chemical, enzymatic or physical process that results in bond breakage. For example, the location can be a nucleotide sequence that is recognized by an endonuclease. Suitable endonucleases and their recognition sequences are well known in the art and in many cases are even commercially available (e.g. from New England Biolabs, Beverley Mass.; ThermoFisher, Waltham, Mass. or Sigma Aldrich, St. Louis Mo.). A particularly useful endonuclease will break a bond in a nucleic acid strand at a site that is 3′-remote to its binding site in the nucleic acid, examples of which include Type II or Type 1Is restriction endonucleases. In some embodiments an endonuclease will cut only one strand in a duplex nucleic acid (e.g. a nicking enzyme). Examples of endonucleases that cleave only one strand include Nt.BstNBI and Nt.Alwl.

In some embodiments, a cleavage site is an abasic site or a nucleotide that has a base that is susceptible to being removed to create an abasic site. Examples of nucleotides that are susceptible to being removed to form an abasic site include uracil and 8-oxo-guanine. Abasic sites can be created by hydrolysis of nucleotide residues using chemical or enzymatic reagents. Once formed, abasic sites may be cleaved (e.g. by treatment with an endonuclease or other single-stranded cleaving enzyme, exposure to heat or alkali), providing a means for site-specific cleavage of a nucleic acid. An abasic site may be created at a uracil nucleotide on one strand of a nucleic acid. The enzyme uracil DNA glycosylase (UDG) may be used to remove the uracil base, generating an abasic site on the strand. The nucleic acid strand that has the abasic site may then be cleaved at the abasic site by treatment with endonuclease (e.g. EndolV endonuclease, AP lyase, FPG glycosylase/AP lyase, EndoVIII glycosylase/AP lyase), heat or alkali. In a particular embodiment, the USER™ reagent available from New England Biolabs is used for the creation of a single nucleotide gap at a uracil base in a nucleic acid.

Abasic sites may also be generated at non-natural/modified deoxyribonucleotides other than uracil and cleaved in an analogous manner by treatment with endonuclease, heat or alkali. For example, 8-oxo-guanine can be converted to an abasic site by exposure to FPG glycosylase. Deoxyinosine can be converted to an abasic site by exposure to AlkA glycosylase. The abasic sites thus generated may then be cleaved, typically by treatment with a suitable endonuclease (e.g. EndolV or AP lyase).

Other examples of cleavage sites and methods that can be used to cleave nucleic acids are set forth, for example, in U.S. Pat. No. 7,960,120, which is incorporated herein by reference.

Modified nucleic acid probes (e.g. extended nucleic acid probes) that are released from a solid support can be pooled to form a fluidic mixture. The mixture can include, for example, at least 10, 100, 1×10³, 1×10⁴, 1×10⁵, 1×10⁶, 1×10⁷, 1×10⁸, 1×10⁹ or more different modified probes. Alternatively or additionally, a fluidic mixture can include at most 1×10⁹, 1×10⁸, 1×10⁷, 1×10⁶, 1×10⁵, 1×10⁴, 1×10³, 100, 10 or fewer different modified probes. The fluidic mixture can be manipulated to allow detection of the modified nucleic acid probes. For example, the modified nucleic acid probes can be separated spatially on a second solid support (i.e. different from the bead array and/or adhered solid support from which the nucleic acid probes were released after having been contacted with a biological specimen and modified), or the probes can be separated temporally in a fluid stream.

Modified nucleic acid probes (e.g. extended nucleic acid probes) can be separated on a bead or other solid support in a capture or detection method commonly employed for microarray-based techniques or nucleic acid sequencing techniques such as those set forth previously and/or otherwise described herein. For example, modified probes can be attached to a microarray by hybridization to complementary nucleic acids. The modified probes can be attached to beads or to a flow cell surface and optionally amplified as is carried out in many nucleic acid sequencing platforms. Modified probes can be separated in a fluid stream using a microfluidic device, droplet manipulation device, or flow cytometer. Typically, detection is carried out on these separation devices, but detection is not necessary in all embodiments.

The number of bead-attached oligonucleotides present upon an individual bead can vary across a wide range, e.g., from tens to thousands, or millions, or more. It is generally preferred to pack as many capture oligonucleotides as spatially and sterically (as well as economically) possible onto an individual bead (i.e., thousands, tens of thousands, or more, of oligonucleotides per individual bead), provided that DNA capture from a contacted tissue is optimized. It is contemplated that optimization of the oligonucleotide-per-bead metric can be readily performed by one of ordinary skill in the art.

It is further expressly contemplated that in addition to the above-described sequence features, oligonucleotides of the instant disclosure can possess any number of other art-recognized features while remaining within the scope of the instant disclosure.

### Capture Material

In certain aspects of the instant disclosure, a capture material is employed to associate a bead array with a solid support (e.g., a glass slide). In some embodiments, the capture material is a liquid electrical tape. An exemplary liquid electrical tape of the instant disclosure is Permatex™ liquid electrical tape, which is a weatherproof protectant for wiring and electrical connections. Liquid capture material such as liquid tape can be applied as a liquid, which then dries to a vinyl polymer that resists dirt, dust, chemicals, and moisture, ensuring that applied beads are attached to a capture material-coated slide in a dry condition. Without wishing to be bound by theory, it is believed that one advantage of the instant methods is that the oligonucleotide-coated beads used in certain embodiments of the invention, which are attached to a solid support (e.g., a slide surface via use, e.g., of electrical tape as a capture material) are maintained in a dry state that optimizes transfer of DNA from a section (e.g., a cryosection) of a tissue to a bead-coated surface (again without wishing to be bound by theory, such transfer is currently believed to occur via capillary action at the scale of the microbead-tissue section interface surface). It is believed that this highly efficient and direct transfer of cellular DNA (i.e., the whole or partial genome, mtDNA, viral and/or bacterial DNA, etc. of cells found within sectioned tissues) to microbeads (where each microbead respectively possesses thousands of oligonucleotides capable of capturing oligoribonucleotides, e.g., transcripts) arrayed upon a solid support—where the transfer occurs upon an otherwise dry surface, therefore limiting and/or eliminating diffusive properties—is one feature that imparts the instant methods and compositions with extremely high resolution (i.e., resolution at 10-50 μm spacing across a two-dimensional image of a section) of assessment of the cellular DNA molecules of assayed tissue sections.

It is contemplated that beads of the instant disclosure can be applied to a capture material-coated solid support, either immediately upon deposit of capture material to the solid support, or following an initial drying period for the capture material. Capture materials of the instant disclosure can be applied by any of a number of methods, including brushed onto the solid support, sprayed onto the solid support, or the like, or via submersion of the solid support in the capture material. For certain forms of liquid capture material, use of a brush top applicator can allow coverage without gaps and can enable access to tight spaces, which offers advantages in certain embodiments over forms of capture material (i.e., tape) that are applied in a non-liquid state.

While liquid electrical tape has been exemplified as a capture material for use in the methods and compositions of the instant disclosure, other capture materials are also contemplated for such use, including any art-recognized glue or other reagent that is (a) spreadable and/or depositable upon a solid surface (e.g., upon a slide, optionally a slide that allows for light transmission through the slide, e.g., a microscope slide) and (b) capable of binding or otherwise capturing a population of beads of 1-100 μm size. Exemplary other capture materials that are expressly contemplated include latex such as cis-1,4-polyisoprene and other rubbers, as well as elastomers (which are generally defined as polymers that possess viscoelasticity (i.e., both viscosity and elasticity), very weak inter-molecular forces, and generally low Young's modulus and high failure strain compared with other materials), including artificial elastomers (e.g., neoprene) and/or silicone elastomers. Acrylate polymers (e.g., scotch tape) are also expressly contemplated, e.g., for use as a capture material of the instant disclosure.

### In Situ Sequencing

In certain aspects of the disclosure, in situ sequencing is performed upon a bead array affixed to a surface, which can be performed by any art-recognized mode of parallel (optionally massively parallel) in situ sequencing, examples of which particularly include the previously described SOLiD™ method, which is a sequencing-by-ligation technique that can be performed in situ upon a solid support (refer, e.g., to Voelkerding et al, Clinical Chem., 55-641-658, 2009; U.S. Pat. Nos. 5,912,148; and 6,130,073, which are incorporated herein by reference in their entireties). In certain embodiments of the instant disclosure, such sequencing can be performed upon a bead array present on a standard microscope slide, optionally using a standard microscope fitted with sufficient computing power to track and associate individual sequences during progressive rounds of detection, with their spatial position(s). The instant disclosure also employed custom fluidics, incubation times, enzymatic mixes and imaging setup in performing in situ sequencing.

### Tissue Samples and Sectioning

In some embodiments, a tissue section is employed. The tissue can be derived from a multicellular organism. Exemplary multicellular organisms include, but are not limited to a mammal, plant, algae, nematode, insect, fish, reptile, amphibian, fungi or Plasmodium falciparum. Exemplary species are set forth previously herein or known in the art. The tissue can be freshly excised from an organism or it may have been previously preserved for example by freezing, embedding in a material such as paraffin (e.g. formalin fixed paraffin embedded samples), formalin fixation, infiltration, dehydration or the like. Optionally, a tissue section can be cryosectioned, using techniques and compositions as described herein and as known in the art. As a further option, a tissue can be permeabilized and the cells of the tissue lysed. Any of a variety of art-recognized lysis treatments can be used. Target nucleic acids that are released from a tissue that is permeabilized can be captured by nucleic acid probes, as described herein and as known in the art.

A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g. less than −20° C. In another example, a tissue can be prepared using formalin-fixation and paraffin embedding (FFPE) methods which are known in the art. Other fixatives and/or embedding materials can be used as desired. A fixed or embedded tissue sample can be sectioned, i.e. thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

In some embodiments, a tissue sample will be treated to remove embedding material (e.g. to remove paraffin or formalin) from the sample prior to release, capture or modification of nucleic acids. This can be achieved by contacting the sample with an appropriate solvent (e.g. xylene and ethanol washes). Treatment can occur prior to contacting the tissue sample with a solid support-captured bead array as set forth herein or the treatment can occur while the tissue sample is on the solid support-captured bead array.

Exemplary methods for manipulating tissues for use with solid supports to which nucleic acids are attached are set forth in US Pat. App. Publ. No. 2014/0066318, which is incorporated herein by reference.

The thickness of a tissue sample or other biological specimen that is contacted with a bead array in a method, composition or apparatus set forth herein can be any suitable thickness desired. In representative embodiments, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a tissue sample that is contacted with bead array will be no more than 100 μm, 50 μm, 10 μm, 5 μm, 1 μm, 0.5 μm, 0.25 μm, 0.1 μm or thinner.

A particularly relevant source for a tissue sample is a human being. The sample can be derived from an organ, including for example, an organ of the central nervous system such as brain, brainstem, cerebellum, spinal cord, cranial nerve, or spinal nerve; an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; a sensory organ such as eye, ear, nose, or tongue; or an organ of the integument such as skin, subcutaneous tissue or mammary gland. In some embodiments, a tissue sample is obtained from a bodily fluid or excreta such as blood, lymph, tears, sweat, saliva, semen, vaginal secretion, ear wax, fecal matter or urine.

A sample from a human can be considered (or suspected) healthy or diseased when used. In some cases, two samples can be used: a first being considered diseased and a second being considered as healthy (e.g. for use as a healthy control). Any of a variety of conditions can be evaluated, including but not limited to, cancer, an autoimmune disease, cystic fibrosis, aneuploidy, pathogenic infection, psychological condition, hepatitis, diabetes, sexually transmitted disease, heart disease, stroke, cardiovascular disease, multiple sclerosis or muscular dystrophy. Certain contemplated conditions include genetic conditions or conditions associated with pathogens having identifiable DNA abundance signatures.

### Application of Wash Solution to Bead Array (Optional)

In certain embodiments, a solid support-captured bead array can be washed after exposure of the bead array to a sectioned tissue (optionally, the sectioned tissue is removed prior to or during application of a wash solution). For example, a solid support-captured bead array of the instant disclosure can be submerged in a buffered salt solution (or other stabilizing solution) after contacting the bead array with a sectioned tissue sample. Exemplified buffered salt solutions include saline-sodium citrate (SSC), for example at a NaCl concentration of about 0.2 M to 5 M NaCl, optionally at about 0.5 to 3 M NaCl, optionally at about 1 M NaCl. In addition to SSC, use of other types of buffered solutions is expressly contemplated, including, e.g. PBS, Tris buffered saline and/or Tris buffer, as well as, more broadly, any aqueous buffer possessing a pH between 4 and 10 and salt between 0-1 osmolarity.

Wash solutions can contain various additives, such as surfactants (e.g. detergents), enzymes (e.g. proteases and collagenases), cleavage reagents, or the like, to facilitate removal of the specimen. In some embodiments, the solid support is treated with a solution comprising a proteinase enzyme. Alternatively or additionally, the solution can include cellulase, hemicellulase or chitinase enzymes (e.g. if desiring to remove a tissue sample from a plant or fungal source). In some cases, the temperature of a wash solution will be at least 30° C., 35° C., 50° C., 60° C. or 90° C. Conditions can be selected for removal of a biological specimen while not denaturing hybrid complexes formed between target nucleic acids and solid support-attached nucleic acid probes.

### Sequencing Methods

Some of the methods and compositions provided herein employ methods of sequencing nucleic acids. A number of DNA sequencing techniques are known in the art, including fluorescence-based sequencing methodologies (See, e.g., Birren et al, Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (PCT Publication No WO2006084132, which is incorporated herein by reference in its entirety). In some embodiments, DNA sequencing is achieved by parallel oligonucleotide extension (See, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; US 20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; WO 00018957, which are incorporated herein by reference in their entireties).

Next-generation sequencing (NGS) methods can be employed in certain aspects of the instant disclosure to obtain a high volume of sequence information (such as are particularly required to perform deep sequencing of bead-associated cellular DNAs following capture of such DNAs from treated tissue sections (e.g., treated cryosections)) in a highly efficient and cost effective manner. NGS methods share the common feature of massively parallel, high-throughput strategies, with the goal of lower costs in comparison to older sequencing methods (see, e.g., Voelkerding et al, Clinical Chem., 55: 641-658, 2009; MacLean et al, Nature Rev. Microbiol, 7-287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina, and the Supported Oligonucleotide Ligation and Detection (SOLiD™) platform commercialized by Applied Biosystems. Non-amplification approaches, also known as single-molecule sequencing, are exemplified by the HeliScope platform commercialized by Helicos Biosciences, SMRT sequencing commercialized by Pacific Biosciences, and emerging platforms marketed by VisiGen and Oxford Nanopore Technologies Ltd.

In pyrosequencing (U.S. Pat. Nos. 6,210,891; 6,258,568, which are incorporated herein by reference in their entireties), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al, Clinical Chem., 55-641-658, 2009; MacLean et al, Nature Rev. Microbiol, 7:287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55: 641-658, 2009; U.S. Pat. Nos. 5,912,148; and 6,130,073, which are incorporated herein by reference in their entireties) can initially involve fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

### Imaging/Image Assembly

With spatial barcodes of individual beads identified, and with sequences of those DNAs captured by individual bead-attached oligonucleotides (capture probes) also identified, high-resolution images that localize sites of DNA abundance can be readily constructed in silico. In certain embodiments, the spatial locations of a large number of beads within an array can first be assigned to an image location, with all associated DNA sequence data also assigned to that position (optionally, effectively de-coupling the spatial barcode from the array/matrix of DNA sequence information associated with a given site/bead, once the spatial barcode has been used to assign the DNA sequence information to an array position). High resolution images representing the extent of capture of individual or grouped DNAs across the various spatial positions of the arrays can then be generated using the underlying DNA sequence information (which was at least originally bead-associated). Images (i.e., pixel coloring and/or intensities) can be adjusted and/or normalized using any (or any number of) art-recognized technique(s) deemed appropriate by one of ordinary skill in the art.

In certain embodiments, a high-resolution image of the instant disclosure is an image in which discrete features (e.g., pixels) of the image are spaced at 50 μm or less. In some embodiments, the spacing of discrete features within the image is at 40 μm or less, optionally 30 p.m or less, optionally 20 μm or less, optionally 15 μm or less, optionally 10 μm or less, optionally 9 μm or less, optionally 8 μm or less, optionally 7 μm or less, optionally 6 μm or less, optionally 5 μm or less, optionally 4 μm or less, optionally 3 μm or less, optionally 2 μm or less, or optionally 1 μm or less.

Images can be obtained using detection devices known in the art. Examples include microscopes configured for light, bright field, dark field, phase contrast, fluorescence, reflection, interference, or confocal imaging. A biological specimen can be stained prior to imaging to provide contrast between different regions or cells. In some embodiments, more than one stain can be used to image different aspects of the specimen (e.g. different regions of a tissue, different cells, specific subcellular components or the like). In other embodiments, a biological specimen can be imaged without staining.

In particular embodiments, a fluorescence microscope (e.g. a confocal fluorescent microscope) can be used to detect a biological specimen that is fluorescent, for example, by virtue of a fluorescent label. Fluorescent specimens can also be imaged using a nucleic acid sequencing device having optics for fluorescent detection such as a Genome Analyzer®, MiSeq®, NextSeq® or HiSeq® platform device commercialized by lllumina, Inc. (San Diego, Calif.); or a SOLiD™ sequencing platform commercialized by Life Technologies (Carlsbad, Calif.). Other imaging optics that can be used include those that are found in the detection devices described in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and US Pat. App. Publ. No. 2008/0108082, each of which is incorporated herein by reference.

An image of a biological specimen can be obtained at a desired resolution, for example, to distinguish tissues, cells or subcellular components. Accordingly, the resolution can be sufficient to distinguish components of a biological specimen that are separated by at least 0.5 μm, 1 μm, 5 μm, 10 μm, 50 μm, 100 μm, 500 μm, 1 mm or more. Alternatively or additionally, the resolution can be set to distinguish components of a biological specimen that are separated by at least 1 mm, 500 μm, 100 μm, 50 μm, 10 μm, 5 μm, 1 μm, 0.5 μm or less.

A method set forth herein can include a step of correlating locations in an image of a biological specimen with barcode sequences of nucleic acid probes that are attached to individual beads to which the biological specimen is, was or will be contacted. Accordingly, characteristics of the biological specimen that are identifiable in the image can be correlated with the nucleic acids that are found to be present in their proximity. Any of a variety of morphological characteristics can be used in such a correlation, including for example, cell shape, cell size, tissue shape, staining patterns, presence of particular proteins (e.g. as detected by immunohistochemical stains) or other characteristics that are routinely evaluated in pathology or research applications. Accordingly, the biological state of a tissue or its components as determined by visual observation can be correlated with molecular biological characteristics as determined by spatially resolved nucleic acid analysis.

A solid support upon which a biological specimen is imaged can include fiducial markers to facilitate determination of the orientation of the specimen or the image thereof in relation to probes that are attached to the solid support. Exemplary fiducials include, but are not limited to beads (with or without fluorescent moieties or moieties such as nucleic acids to which labeled probes can be bound), fluorescent molecules attached at known or determinable features, or structures that combine morphological shapes with fluorescent moieties. Exemplary fiducials are set forth in US Pat. App. Publ. No. 2002/0150909 A1 or US Pat. App. Ser. No. 14/530,299, each of which is incorporated herein by reference. One or more fiducials are preferably visible while obtaining an image of a biological specimen. Preferably, the solid support includes at least 2, 3, 4, 5, 10, 25, 50, 100 or more fiducial markers. The fiducials can be provided in a pattern, for example, along an outer edge of a solid support or perimeter of a location where a biological specimen resides. In one embodiment, one or more fiducials are detected using the same imaging conditions used to visualize a biological specimen. However if desired separate images can be obtained (e.g. one image of the biological specimen and another image of the fiducials) and the images can be aligned to each other.

### Kits

The instant disclosure also provides kits containing agents of this disclosure for use in the methods of the present disclosure. Kits of the instant disclosure may include one or more containers comprising an agent (e.g., a capture material, such as liquid electrical tape) and/or composition (e.g., a slide-captured bead array) of this disclosure. In some embodiments, the kits further include instructions for use in accordance with the methods of this disclosure. In some embodiments, these instructions comprise a description of administration of the agent to diagnose, e.g., a disease and/or malignancy. In some embodiments, the instructions comprise a description of how to create a tissue cryosection, treat a tissue section with a permeabilizing agent and/or a DNA fragmenting agent, form a spatially-defined (or simply spatially definable, pending performance of a step that defines the spatial resolution of the bead array) bead array, contact a tissue cryosection with a spatially-defined bead array and/or obtain captured, tissue cryosection-derived DNA sequence from the spatially-defined bead array. The kit may further comprise a description of selecting an individual suitable for treatment based on identifying whether that subject has a certain pattern of DNA abundance of one or more sequences in a cryosection sample.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for staging a cryosection and/or diagnosing a specific DNA abundance pattern in a cryosection. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Preparation of Bead Capture Slide Surface

Slide surfaces for bead deposit (bead capture slides) were prepared by coating a microscope slide in silicone, and the microscope slides were then spin coated with liquid electrical tape. Slides were allowed to dry. Once dry, a rubber gasket having a hole diameter of 3 mm was placed upon the slide surface. 100,000 beads suspended in water were then added, and the bead-contacted slide was then spun down in swinging bucket centrifuge for 60 minutes until the water completely dried. Bead-captured slides were then washed with water three times to remove residual beads, which yielded a bead monolayer. Bead slides were then stored dry at 4° C. until used.

Puck preparation, sequencing, image processing, and basecalling were performed as described in Rodrigues, Stickels et al. (Science 363(6434): 1463-1467) and infra. However, a different oligonucleotide was coupled to the beads to allow for capture of DNA (as opposed to RNA): 5′-TTTT-PCT-GCCGCTACACGACGCTCTTCCGATCTJJJJJJJ JTCTTCAGCGTTCCCGAGAJJJJJJJ GCTCG GACACATGGGCG-3′ (SEQ ID NO: 1), where “J” represents a degenerate nucleotide that is consistent across all oligonucleotides on a given bead (as opposed to UMIs, which would be truly unique for each capture oligonucleotide employed (differing from oligonucleotide to oligonucleotide)—the sum of the 14 “J” residues shown form the spatial barcode employed herein.

### Treatment of Tissue Sections and Transfer of Cellular DNA to Bead Surface

A 10-micron coronal section of fresh-frozen mouse brain was cut and treated with permeabilizing agent and subjected to tagmentation, using the following procedures:

### Transposome Assembly

Fresh transposomes were prepared on the day of tagmentation. The following oligos (ordered from IDT) were combined at a 1:1:2 molar ratio, each at 100 μM starting concentration: adapter 1 (/5Phos/GAGCTTTGCTAACGGTCGAG AGATGTGTATAAGAGACAG; SEQ ID NO: 2), adapter 2 (CTTACGGATGTTGCACCAGCAGATGTGTATAAGAGACAG; SEQ ID NO: 3), and mosaic end (/5Phos/CTGTCTCTTATACA /3ddC/; SEQ ID NO: 4). In the presence of 50 mM NaCl, 10 mM Tris-HCl pH 7, this mixture is heated at 85° C. for 3 minutes and then cooled to 20° C. over approximately 1-1.5 hours (1% ramp). The annealed adaptors were then diluted with 100% glycerol at a 1:1 ratio. This adapter-glycerol mix can be stored at −20° C. for up to 6 months. Separately, 100 μl of Tn5 dilution buffer was prepared fresh by mixing 50 μl of 100% glycerol, 5 μl of 1 M Tris pH 7.5, 2 μl of 5 M NaCl, 2 μl of 5 mM EDTA, 1 μl of 100 mM DTT, 1 μl of 10% NP-40 and 39 μl of water. 25 μl of unloaded Tn5 enzyme (gift from Feng Zhang Lab) was then mixed with 25 μl of Tn5 dilution buffer and 50 μl of the annealed adaptor-glycerol mix was left to incubate at for 30 minutes at room temperature, and stored at −20° C. until tagmentation.

### Tissue Preparation and Tagmentation

Unless otherwise specified, all reagents were diluted into PBS, washes were performed with PBS, and steps were carried out at room temperature. Fresh frozen tissue was sectioned either on a poly-lysine-coated glass slide (workflow A), or onto a + slide (puck) (workflow B), and fixed with 1% paraformaldehyde for 10 minutes, quenched with 250 mM Tris-HCl pH 8, washed twice, and kept at 4° C. overnight. For the Assay for Transposase-Accessible Chromatin (ATAC), the tissue was permeabilized by incubating with 0.5% Triton-X for 30 min. For whole genome preparation, two methods were used to permeabilize the tissue and deplete nucleosomes: 1) Tissue was treated with 0.5% Triton-X for 30 min, washed, and incubated with 0.1N HCl for 5 min. 2) Tissue was treated with SDS (range from 0.8% -8%, diluted into water) for 10 min at 60C, followed by 1.5% Triton-X (diluted in water) for 10 minutes, washed, and optionally incubated with proteinase K (range from 1−20 ug/mL) for 10 minutes at 37C. Subsequently, the tissue was washed, pre-incubated with tagmentation buffer (5% DMF, 10 mM Tris pH 7.5, 5 mM MgCl2, 0.3× PBS) and then tagmented at 37° C. for 1 hour with the preloaded Tn5 (diluted 1:10 into tagmentation buffer).

### Linking Spatial Barcodes to Genomic Fragments

Workflow A: The puck was hybridized with 10 μM splint oligo (CGTTAGCAAAGCTC CGCCCATGTGTC (SEQ ID NO: 5), in 4× SSC) at 37° C. for 1 hour and washed in 4× SSC at room temperature. Using a microarray hybridization chamber (Agilent G2534A), the puck was pressed against the tissue. The puck-tissue block was then exposed to 300 nm LED light for 3 minutes, which photocleaved the oligos from the beads, and then incubated for 1 hour at 37° C.

Workflow B: The tissue-puck sample was hybridized with 500 nM of splint oligo (in 4× SSC) for 2h at 37° C., washed twice with 4× SSC at room temperature, exposed to blue light inside the third 4× SSC wash to cleave the beads, and incubated for 1 hour at 37° C.

Subsequently, the sample was washed twice with 4× SSC, pre-incubated with T4 ligase buffer, then ligated with T4 ligase (12 U/μL in T4 buffer) for 1 hour at room temperature, and finally digested with Proteinase K (400 μg/mL in 50 mM Tris pH 8, 50 mM NaCl, 0.2% SDS) overnight at 37° C., or for 4 hours at 55° C. The DNA was purified with Machery-Nagel NuceoSpin Gel and PCR Clean-up kit.

### Library Amplification

The library was amplified as described in Buenrostro et al. (Curr Protoc Mol Biol 2015; 109: 21.29.1-21.29.9), with the modification that annealing was done at 65° C.; forward primer (Truseq) 5′-AATGATACGGCGACCACCGAGATCTACAC-3′ (SEQ ID NO: 6)
(i5 barcode) ACACTCTTTCCCTACACGACGCTCTTCCGATC*T-3′ (SEQ ID NO: 7); reverse primer 5′-CAAGCAGAAGACGGCATACGAGAT-3′ (SEQ ID NO: 8)
(i7 barcode) GTTGGCACCAGGCTTACGGATGTTGCACCAGC-3′ (SEQ ID NO: 9).
The amplified library was cleaned with AMPure XP beads, its concentration was quantified with KAPA Library Quantification Kit, and the library was loaded onto an Illumina® Nextseq with the following custom sequencing primers and read structure:

Read 1: 57 bp,
(SEQ ID NO: 10)
GCTTTGCTAACGGTCGAG AGATGTGTATAAGAGACAG
Read 2: 57 bp,
(SEQ ID NO: 11)
CGGATGTTGCACCAGC AGATGTGTATAAGAGACAG
Index 1: 8 bp,
(SEQ ID NO: 12)
CTCGACCGTTAGCAAAGCTCCGCCCATGTGTC
Index 2: 44 bp,
(SEQ ID NO: 13)
GCTGGTGCAACATCCGTAAGCCTGGTGCCAAC

Mapping of the bead spatial barcodes from in situ and Illumina® sequencing was performed as described in Rodrigues, Stickels et al.

### Example 2: Stable Association of Individually Barcode-Tagged Microbeads with a Glass Slide Provided a High-Resolution Array for Cellular DNA Capture

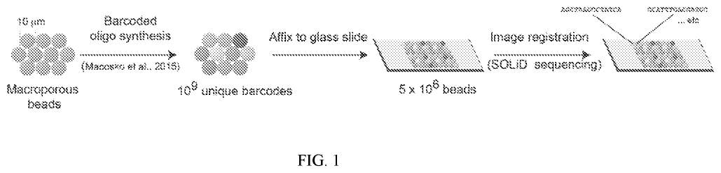

A large number of 10 μm beads that possessed unique nucleic acid barcodes were prepared via methods as described previously (e.g., as set forth in WO 2016/040476). Specifically, to generate a population of beads possessing individual barcodes that could be used for identification of an individual bead's position when arranged in a two-dimensional array as presently exemplified, polynucleotide synthesis was performed upon the surface of the beads in a pool-and-split fashion such that in each cycle of synthesis the beads were split into subsets that were subjected to different chemical reactions; and then this split-pool process was repeated in multiple cycles, to produce a combinatorially large number (approaching 4^{π}) of distinct nucleic acid barcodes (FIG. 1). Nucleotides were chemically built onto the bead material in a high-throughput manner, and the bead population that was used possessed approximately a billion (10⁹) unique bead-specific barcodes. After on-bead oligonucleotide synthesis, a glass slide was employed as a solid support for generation of an array of barcoded beads. To provide a capture material-coated surface for the bead array, the glass slide was initially coated with liquid electrical tape (applied as a liquid, the liquid tape dried to a vinyl polymer).

Barcoded beads as described above were applied to the capture material-coated slide, generating an array of beads in a dry condition (excess, non-captured beads were removed from the slide, thereby producing a single layer of captured beads). Because individually barcoded beads were deposited upon the capture material-coated surface in no pre-defined order, in situ sequencing of the bead array while captured upon the slide was performed, using the previously described SOLiD™ method (a sequencing-by-ligation technique that can be performed in situ upon a solid support—refer, e.g., to Voelkerding et al, Clinical Chem., 55-641-658, 2009; U.S. Pat. Nos. 5,912,148; 6,130,073, which are incorporated herein by reference in their entireties), thereby associating a bead's spatial barcode sequence with the two-dimensional location of that bead within the two-dimensional, slide-captured bead array (FIG. 1).

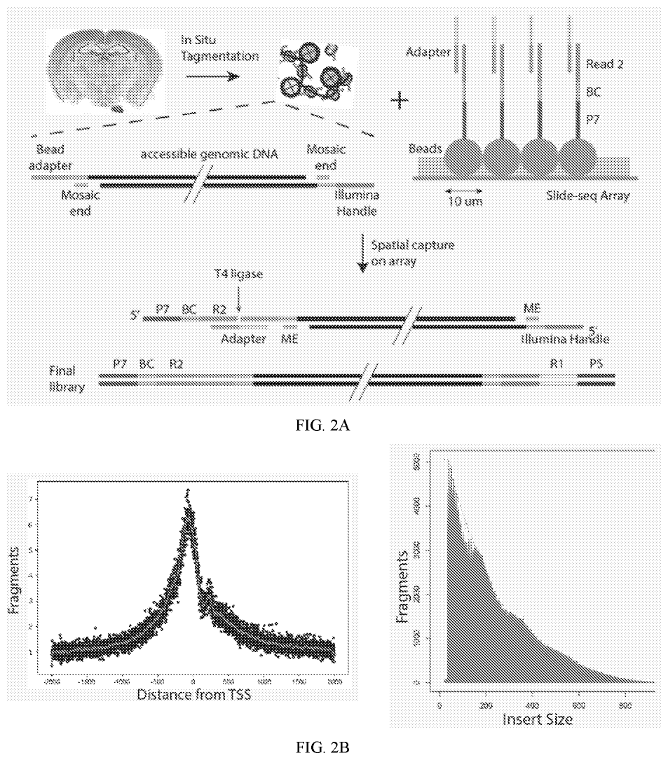

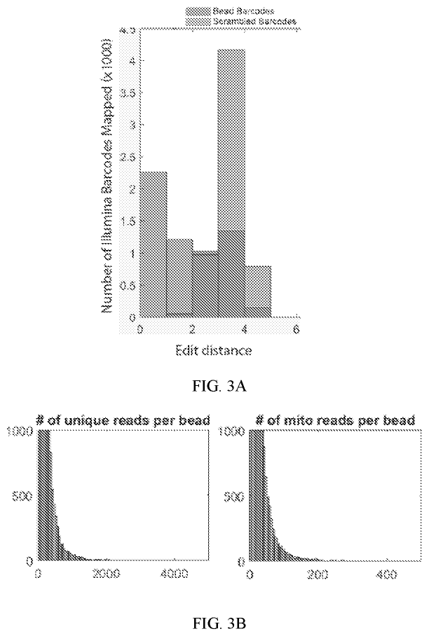

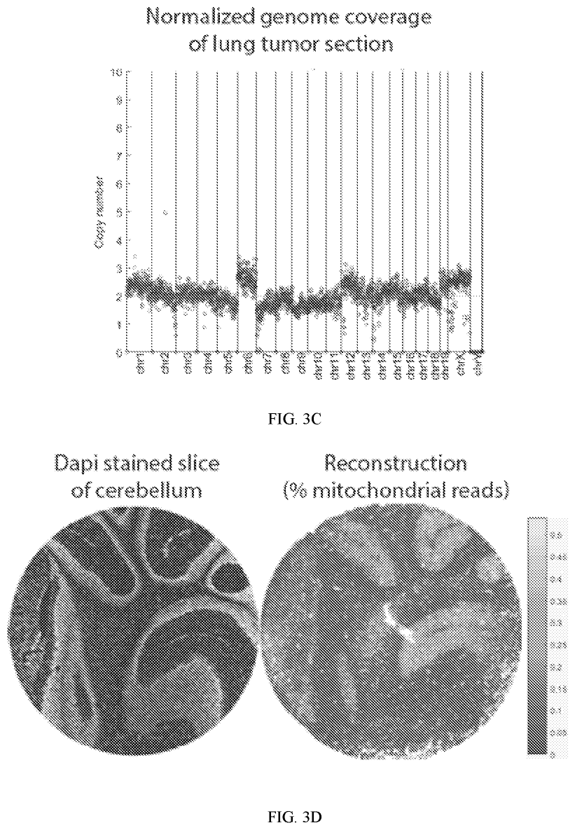

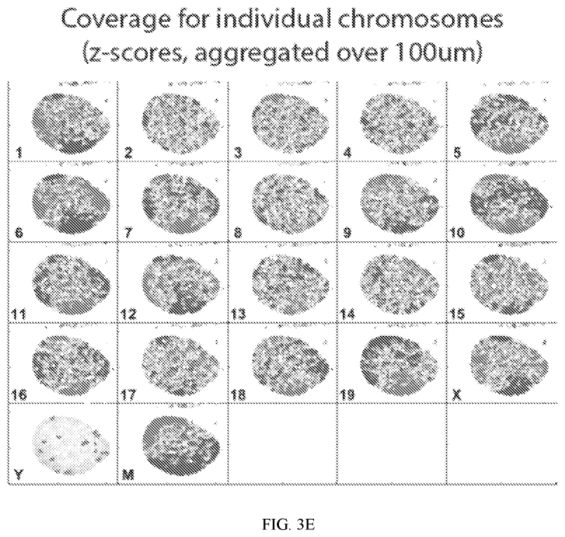

The oligonucleotide-coated microbeads were thus attached to a glass slide surface as a two-dimensional solid support, and bead-attached oligonucleotide sequences were obtained within the spatial barcode sequence region for purpose of registering the respective locations of microbeads assorted throughout the array—in certain examples of bead-attached oligonucleotide sequences, each oligonucleotide respectively includes: a site of attachment (optionally, a cleavable site of bead attachment), an initial adapter sequence (e.g., a P7 adapter/PCR primer sequence of FIG. 2A); a spatial barcode that is unique (or sufficiently unique) to each bead (as described above and as previously as noted); and a sequence capable of capturing tagmented cellular DNA sequences (in FIG. 2A, this capture sequence is a “Read 2” sequence). This high-resolution bead array was then used for cellular DNA capture from treated sample tissue, which was prepared as described infra. For initial tagmentation preparations as shown in FIG. 2A, DNA fragments obtained from the process were not uniformly distributed in terms of genomic location, but were enriched at particular locations (of presumably more accessible DNA; FIG. 2B, at left). A profile of observed insert sizes obtained by the instant tagmentation process is shown in FIG. 2B, at right. As shown in FIG. 3A, the proportions of SOLiD™ sequence bead barcodes that matched uniquely to bead barcodes as sequenced by the Illumina™ method differed dramatically between in situ sequenced data, which mostly matched at edit distances of 0 or 1 (<2), whereas random shuffling of SOLiD™ barcodes generated very few matches at edit distances of 0, 1 or 2. It was further identified that the instant tagmentation-mediated approach provided a distribution of unique reads per bead (FIG. 3B, at left), which included mtDNA reads (FIG. 3B, at right).

A permeabilized and tagmented cryosection of a lung tumor was contacted to a bead array as described infra, and regions of copy number variation (CNV) were successfully identified (FIG. 3C). (It is herein specifically noted that as an alternative to using a DNA splint oligonucleotide and T4 ligase to link the tissue DNA fragments to beads, a RNA splint oligonucleotide and a RNA ligase, such as SplintR ligase, can also be used.)

### Example 3: A Glass Slide-Associated Barcode-Tagged Microbead Array Captured Cellular DNAs with Robust Spatial Resolution

A cerebellum tissue sample was obtained from a mouse and was cryosectioned to reveal a cross-section of structures. The cerebellum cryosection was permeabilized and tagmented, then applied to a high-resolution bead array of the above Example (the bead array slide was at room temperature), and transfer of cellular DNA molecules of the cerebellum cryosection to the bead array was allowed to occur. The cerebellum cryosection was then removed, and the slide-captured bead array was submerged in approximately 6× saline-sodium citrate (SSC) buffer, which contained approximately 1 M NaCl, and bulk sequencing of bead-attached oligonucleotides (including sequencing of oligonucleotide-captured DNA molecules, where post-amplification, DNA amplicons were released from the array and then analyzed, for example, by high-throughput next generation sequencing (NGS), such as sequencing-by-synthesis (SBS)) was performed. As shown in FIG. 3D, spatial reconstruction (using barcoded bead-derived oligonucleotide sequences) of cellular mitochondrial DNA reads obtained from the capture bead array identified DNA abundance levels across the tissue section (FIG. 3D, at right) consistent with those observed for DAPI staining (of DNA) in the cerebellum section (FIG. 3D, at left).

Spatial reconstruction of chromosome coverage in a tumor tissue section is shown in FIG. 3E and demonstrates the ability of the technology of the instant disclosure to capture and accurately identify the varying spatial distribution of copy number alterations.

Thus, the instant methods have enabled generation of a high-resolution image that accurately depicts the spatial distribution of expression for bead-captured DNA molecules of a tissue sample.

The current compositions and methods can be used to understand the spatial organization of tissues in health and disease, with exemplary applications including: 1) Use for atlasing efforts to understand the diversity of cell types and their interactions in tissues and organs. 2) Use for studying how cell types change in tissue in response to perturbation and disease. 3) Use for studying post-mortem or clinical samples, and in particular, use to relate histopathological findings in diseased tissue to specific DNA abundance changes in specific cell types.

Application of the current compositions and methods to investigate DNA abundance patterns in tissues, potentially as a diagnostic tool, or as a tool for developing diagnostic assays, or pathological staging, for diseases (e.g., the instant approach can be used to profile many cancer sections (optionally alongside normal control sections), to reveal a spatial DNA abundance signature predictive of disease course and/or treatment response) is also expressly contemplated.

Arriving at the improved methods for spatial assessment of DNA abundance set forth herein involved confronting and overcoming a number of obstacles, including the following examples of approaches that were identified to be sub-optimal and/or non-working:
During tagmentation, replacing the mosaic ends directly with the bead oligonucleotide yielded sub-optimal results. In particular, further to Wang et al. (Nature Protocols 8, 2022−2032), displacement of the mosaic end after tagmentation was attempted, with the intent of letting the bead oligonucleotide hybridize to genomic fragments directly. However, this approach did not yield diverse libraries.
Attempts to recover more fragments via use of the Chen et al. (Science 356 (6334):

189-194) protocol for linear amplification of tagmented DNA proved refractory. The in vitro transcribed RNA produced by such a method would, in theory, then be captured with slide-seq beads, which are coated with poly-dT containing oligos. However, this approach did not yield good libraries.
Enhanced bead oligonucleotide design provided significant benefits. Initially, the bead was coupled to oligonucleotides that possessed the structure [p5—spatial barcode-Read2]5′-CAAGCAGAAGACGGCATACGAGATJJJJJJJJJJJJJGTTGGCACCAGGCTTACG GATGTTGCACCAGC-3′ (SEQ ID NO: 14). While libraries were successfully obtained using this oligonucleotide, the spatial barcodes from Illumina® and in situ sequencing did not match.
Tissue and puck set-up was improved for the current disclosure. It was initially reasoned that for optimal spatial resolution, first hybridizing the puck with the bridge oligo, then placing the tissue section on top, and then tagmenting the tissue on top of the puck would be best. However, this approach did not yield good libraries (without wishing to be bound by theory, this was likely because the transposase predominantly tagmented the double stranded regions on the puck).
Yield of DNA molecules from tissue was found to be much better without EDTA treatment (EDTA treatment of tissue after tagmentation was initially attempted for removal of bound Tn5 molecules from the genomic fragments (which, it was speculated, might make the DNA more accessible to ligation with bead oligos)).
It has often been observed in other tagmentation approaches that naive cells can yield more complex libraries from Tn5 transposition than fixed cells. However, when attempts were made herein to isolate DNA from unfixed tissue, tissue integrity suffered through the process.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for obtaining spatially-resolvable DNA abundance data from a tissue sample comprising:
(i) obtaining a tissue sample from a subject;
(ii) preparing a section of the tissue sample;
(iii) contacting the section of the tissue sample with a permeabilizing agent and a DNA fragmenting agent, thereby producing a treated sectioned tissue sample;
(iv) obtaining a solid support;
(v) contacting the solid support with a capture material, thereby forming a capture material-coated solid support;
(vi) contacting the capture material—coated solid support with a population of 1-100 μm diameter beads, wherein each bead has at least 1000 attached oligonucleotides and wherein at least 1000 attached oligonucleotides of each bead each comprises: (a) a bead identification sequence that is common to all at least 1000 oligonucleotides on each bead and (b) a target DNA-specific capture sequence, thereby forming a subpopulation of beads attached to the solid support;
(vii) identifying the bead identification sequence and associated two-dimensional position on the solid support of individual beads of the subpopulation of beads attached to the solid support;
(viii) contacting the subpopulation of 1-100 μm diameter beads captured upon the solid support with the treated sectioned tissue sample; and
(ix) obtaining the sequences of a population of target DNA molecules bound to the bead oligonucleotides and an associated bead identification sequence for each target DNA molecule sequenced,
thereby obtaining spatially-resolvable DNA abundance data from the tissue sample.

2. The method of claim 1, wherein the permeabilizing agent is selected from the group consisting of Triton X-100, NP-40, methanol, acetone, Tween 20, saponin, Leucoperm™, and digitonin.

3. The method of claim 2, wherein the permeabilizing agent is Triton X-100 at a concentration between about 0.01% and about 5%.

4. The method of claim 1, wherein the contacting of the section of the tissue sample with the permeabilizing agent is performed for a duration of time between about 0.1 minute and about 30 minutes.

5. The method of claim 1, wherein the DNA fragmenting agent is selected from the group consisting of a transposase; H₂O₂; sonication; and a DNase, optionally wherein the DNase is a restriction endonuclease.

6. The method of claim 5, wherein the DNA fragmenting agent comprises a Tn5 transposase enzyme, optionally wherein the Tn5 transposase enzyme is present in a tagmentation buffer comprising adapater and mosaic oligonucleotides.

7. The method of claim 6, wherein the Tn5 enzyme in tagmentation buffer comprising adapater and mosaic oligonucleotides contacts the sectioned tissue sample for about 20 minutes to about 16 hours, optionally wherein the Tn5 enzyme in tagmentation buffer comprising adapater and mosaic oligonucleotides contacts the sectioned tissue sample at between about 25° C. and about 55° C.

8. The method of claim 1, wherein the section of the tissue sample is a cryosection.

9. The method of claim 1, wherein the target DNA molecules are selected from the group consisting of genomic DNA molecules, mitochondrial DNA (mtDNA) molecules, viral DNA molecules (optionally retroviral DNA molecules or AAV DNA molecules) and bacterial DNA molecules.

10. The method of claim 1, wherein the target DNA molecules are genomic DNA molecules, optionally wherein the genomic DNA molecules are enriched for accessible chromatin sequences, as compared to inaccessible chromatin sequences (e.g., genomic DNA sequences associated with nucleosomes and/or other forms of condensed chromatin).

11. The method of claim 1, wherein the permeabilizing agent is about 0.1% to about 0.5% Triton X-100, optionally wherein the contacting of the section of the tissue sample with the Triton X-100 is performed for a duration of time between about 10 minutes and about 60 minutes.

12. The method of claim 1, wherein step (iii) further comprises contacting the section of the tissue sample with a nucleosome disrupting agent, optionally wherein the nucleosome disrupting agent is selected from the group consisting of HCl, SDS, and a protease/proteinase.

13. The method of claim 12, wherein the nucleosome disrupting agent is about 0.01N to about 0.5N HCl, optionally wherein contacting the section of the tissue sample with HCl is performed for about one to about 10 minutes.

14. The method of claim 12, wherein the nucleosome disrupting agent is about 0.1% to about 10% SDS, optionally wherein contacting the section of the tissue sample with SDS is performed for about 5 to about 15 minutes, optionally at about 30° C. to about 70° C.

15. The method of claim 1, wherein step (iii) further comprises contacting the section of the tissue sample with proteinase K, optionally about one to about 20 μg/m1 proteinase K, optionally for a duration of time of about 5 to about 15 minutes, optionally at about 25° C. to about 50° C.

16. The method of claim 1, wherein the tissue sample is obtained from a tissue selected from the group consisting of brain, lung, liver, kidney, pancreas, heart, and gastrointestinal (GI) tract.

17. The method of claim 1, wherein:
the tissue sample is obtained from a tumor;
the subject is a mammal, optionally a human;
the spatially-resolvable DNA abundance data identifies regions of copy number variation (CNV) in the section of the tissue sample, optionally regions of genetic amplification in the tissue sample, optionally regions of trisomy;
the spatially-resolvable DNA abundance data identifies regions of aneuploidy in the section of the tissue sample;
the spatially-resolvable DNA abundance data identifies regions of related cellular lineage in the section of the tissue sample;
the tissue sample is fixed, optionally wherein the tissue sample is fixed with paraformaldehyde, optionally for 5-25 minutes, optionally quenched with 100-500mM Tris-HCl;
the solid support is a slide, optionally the solid support is a glass slide;
the capture material is applied as a liquid, optionally wherein the capture material is applied using a brush or aerosol spray, optionally wherein the capture material is a liquid electrical tape, optionally wherein the capture material dries to form a vinyl polymer, optionally wherein the vinyl polymer is polyvinyl hexane;
the 1-100 μm diameter beads comprise porous polystyrene, porous polymethacrylate and/or polyacrylamide;
the beads are 1-40 μm diameter beads, optionally wherein the beads are 10 μm beads;
the step of (vii) identifying the bead identification sequence and associated two-dimensional position on the solid support of individual beads of the subpopulation of beads attached to the solid support comprises performance of a sequencing-by-ligation technique;
the subpopulation of 1-100 μm diameter beads captured upon the solid support in step (viii) is maintained at a temperature between about 4° C. and about 30° C., optionally at about 25° C.;
step (viii) further comprises contacting the subpopulation of 1-100 μm diameter beads captured upon the solid support with a wash solution, optionally with a saline solution, optionally with a solution comprising between about 1M and about 3M NaCl, optionally with a saline-sodium citrate buffer comprising between about 1M and about 3M NaCl;
step (ix) obtaining the sequences of a population of DNA molecules bound to the bead oligonucleotides and an associated bead identification sequence for each target DNA molecule sequenced comprises a next-generation sequencing approach, optionally wherein the next-generation sequencing approach is selected from the group consisting of solid-phase, reversible dye-terminator sequencing; massively parallel signature sequencing; pyro-sequencing; sequencing-by-ligation; ion semiconductor sequencing; Nanopore sequencing and DNA nanoball sequencing, optionally wherein the next-generation sequencing approach is solid-phase, reversible dye-terminator sequencing;
the bead identification sequence and associated two-dimensional position on the solid support of individual beads of the subpopulation of beads attached to the solid support is registered in a computer;
further comprising step (x) generating an image of the tissue sample that depicts the location(s) and relative abundance of one or more captured target DNAs within the sample, optionally wherein the image is a two-dimensional image; and/or
the spatially-resolvable DNA abundance data identifies one or more features in the section of the tissue sample selected from the group consisting of:
a) mitochondrial lineage;
b) epigenetic modification(s) and/or difference(s) in regions of chromatin accessibility across the section of the tissue sample;
c) regions of monoallelic gene expression and/or gene dosage;
d) cellular delivery of CRISPR/Cas9 plasmid(s) and/or gels, TALEN plasmid(s) and/or gels, viral vectors (e.g., AAV), expression vectors/plasmids and/or other gene therapy agents/payloads;
e) histology; and/or
f) response in the tissue to a pre-administered drug or other agent, as compared to an appropriate control.

18-33. (canceled)

34. A method selected from the group consisting of:
A method for providing access to cellular DNA of a tissue sample in situ, the method comprising:
(i) obtaining a tissue sample from a subject;
(ii) preparing a section of the tissue sample; and
(iii) contacting the section of the tissue sample with a permeabilizing agent and a DNA fragmenting agent, thereby providing access to cellular DNA of the tissue sample in situ; and
An improved method for obtaining spatial DNA abundance data from a tissue sample, wherein the improvement comprises:
i) obtaining a section of a tissue sample;
ii) contacting the section of the tissue sample with a permeabilizing agent and a DNA fragmenting agent, thereby producing a treated section of the tissue sample; and
iii) contacting the treated section of the tissue sample with a capture material—coated solid support comprising a population of beads wherein each bead has at least 1000 attached oligonucleotides and wherein each of the at least 1000 attached oligonucleotides of each bead comprises: (a) a bead identification sequence that is common to all at least 1000 oligonucleotides on each bead and (b) a target DNA-specific capture sequence, thereby obtaining spatial DNA abundance data from the tissue sample.

35. The method of claim 34, wherein:
the section of the tissue sample is a cryosection;
the method further comprises (iv) contacting the section of the tissue sample with an array of DNA capture probes, optionally with a bead array comprising DNA capture probes, optionally wherein the bead array comprising DNA capture probes is attached to a solid support, optionally wherein the spatial locations upon the solid support of a selection of beads of the bead array are known upon contact of the bead array with the section of the tissue sample;
the target DNA-specific capture sequence anneals to an exogenously introduced adapter sequence in the target DNA of the section of the tissue sample;
the target DNA-specific capture sequence anneals to one or more pre-selected target DNA sequences within the section of the tissue sample, optionally wherein the target DNA-specific capture sequence anneals to one or more pre-selected endogenous target DNA sequences within the section of the tissue sample and/or the target DNA-specific capture sequence anneals to one or more viral or bacterial target DNA sequences within the section of the tissue sample, optionally wherein the viral target DNA sequences are adeno-associated virus (AAV) target DNA sequences; and/or
one or more non-DNA macromolecules or small molecules are extracted and/or identified in the same section of the tissue sample or in one or more adjacent section(s) of the tissue sample.

36-42. (canceled)

43. A kit comprising:
(i) a permeabilizing agent;
(ii) a DNA fragmenting agent;
(iii) a capture material—coated solid support with a population of 1-100 μm diameter beads, wherein each bead has at least 1000 attached oligonucleotides and wherein at least 1000 attached oligonucleotides of each bead each comprises: (a) a bead identification sequence that is common to all at least 1000 oligonucleotides on each bead and (b) a target DNA-specific capture sequence, thereby forming a subpopulation of beads attached to the solid support;
and instructions for its use.


SEQUENCE LISTING (14 sequences)

SEQ ID NO: 1 - DNA, 82 bp, Artificial
  Synthetic
  misc_feature (5)..(7): n is a, c, g, or t
  misc_feature (34)..(40): n is a, c, g, or t
  misc_feature (59)..(65): n is a, c, g, or t
ttttnnngccgctacacgacgctcttccgatctnnnnnnntcttcagcgttcccgagannnnnnngctcggacacatgggcg

SEQ ID NO: 2 - DNA, 39 bp, Artificial
  Synthetic
gagctttgctaacggtcgagagatgtgtataagagacag

SEQ ID NO: 3 - DNA, 39 bp, Artificial
  Synthetic
cttacggatgttgcaccagcagatgtgtataagagacag

SEQ ID NO: 4 - DNA, 14 bp, Artificial
  Synthetic
ctgtctcttataca

SEQ ID NO: 5 - DNA, 26 bp, Artificial
  Synthetic
cgttagcaaagctccgcccatgtgtc

SEQ ID NO: 6 - DNA, 29 bp, Artificial
  Synthetic
aatgatacggcgaccaccgagatctacac

SEQ ID NO: 7 - DNA, 33 bp, Artificial
  Synthetic
acactctttccctacacgacgctcttccgatct

SEQ ID NO: 8 - DNA, 24 bp, Artificial
  Synthetic
caagcagaagacggcatacgagat

SEQ ID NO: 9 - DNA, 31 bp, Artificial
  Synthetic
ttggcaccaggcttacggatgttgcaccagc

SEQ ID NO: 10 - DNA, 37 bp, Artificial
  Synthetic
gctttgctaacggtcgagagatgtgtataagagacag

SEQ ID NO: 11 - DNA, 35 bp, Artificial
  Synthetic
cggatgttgcaccagcagatgtgtataagagacag

SEQ ID NO: 12 - DNA, 32 bp, Artificial
  Synthetic
ctcgaccgttagcaaagctccgcccatgtgtc

SEQ ID NO: 13 - DNA, 32 bp, Artificial
  Synthetic
gctggtgcaacatccgtaagcctggtgccaac

SEQ ID NO: 14 - DNA, 68 bp, Artificial
  Synthetic
  misc_feature (25)..(36): n is a, c, g, or t
caagcagaagacggcatacgagatnnnnnnnnnnnngttggcaccaggcttacggatgttgcaccagc